(19) United States
(12) Patent Application Publication
(10) Pub. No.: US 2025/0345419 A1
(43) Pub. Date: Nov. 13, 2025

(54) TREATING CHEMORESISTANT CANCERS WITH NOTCH3 INHIBITORS

(71) Applicant: Mayo Foundation for Medical Education and Research, Rochester, MN (US)

(72) Inventor: Antonio B. D'Assoro, Rochester, MN (US)

(21) Appl. No.: 18/867,114

(22) PCT Filed: May 19, 2023
(86) PCT No.: PCT/US2023/022973

§ 371 Date: Nov. 19, 2024

Related U.S. Application Data
(60) Provisional application No. 63/344,513, filed on May 20, 2022.
(60) Provisional application No. 63/398,072, filed on Aug. 15, 2022.

(51) Int. Cl. A61K 39/395; A61K 31/337; A61K 39/00; A61P 35/00; C07K 16/28

(52) U.S. Cl. CPC A61K 39/39558; A61K 31/337; A61P 35/00; C07K 16/28; A61K 2039/505; A61K 2039/545; C07K 2317/24; C07K 2317/73; C07K 2317/76; C07K 2317/92

(57) ABSTRACT

Methods and materials for treating mammals identified as having a cancer that is resistant to chemotherapy are provided herein. Also provided herein are methods and materials for identifying mammals having cancer that is resistant to treatment with a chemotherapeutic agent as being likely to respond to subsequent treatment with the chemotherapeutic agent.

### CROSS-REFERENCE TO RELATED APPLICATIONS

This application claims priority from U.S. Provisional Application Ser. No. 63/398,072, filed Aug. 15, 2022, and U.S. Provisional Application Ser. No. 63/344,513, filed May 20, 2022. The disclosures of the prior applications are considered part of (and are incorporated by reference in) the disclosure of this application.

### SEQUENCE LISTING

This application contains a Sequence Listing that has been submitted electronically as an XML file named “07039-2136WO1.XML.” The XML file, created on May 19, 2023, is 5,459 bytes in size. The material in the XML file is hereby incorporated by reference in its entirety.

### TECHNICAL FIELD

This document relates to methods and materials for treating mammals having cancer that is resistant to chemotherapy, and to methods and materials for identifying mammals having chemoresistant cancer as being likely to respond to subsequent treatment with chemotherapy.

### BACKGROUND

NOTCH is an evolutionarily conserved signaling pathway that plays a critical role in embryonic development, cellular proliferation, differentiation, and apoptosis (Fasoulakis et al., Adv Exp Med Biol., 1287:169-181, 2021; and Penton et al., Semin Cell Dev Biol., 23(4):450-457, 2012). Canonical NOTCH signaling consists of four NOTCH receptors (NOTCH 1, NOTCH2, NOTCH3, and NOTCH4) and their ligands (Reichrath and Reichrath, Adv Exp Med Biol., 1218:159-187. 2020). Following ligand binding, γ-secretase complex performs an intra-membrane cleavage, releasing the NOTCH-intracellular domain (ICD) that translocates to the nucleus and interacts with Co-Activators and CLS complex.

Epithelial to Mesenchymal Transition (EMT) drives the conversion from a polarized epithelial phenotype to an elongated fibroblastoid-like phenotype that typifies the morphology of highly invasive cancer cells (Mani et al., Proc Natl Acad Sci USA., 104(24):10069-10074, 2007). Aberrant activation of NOTCH signaling can promote an invasive tumor phenotype through activation of EMT-mediated cancer cell plasticity and tumor stemness reprogramming in a variety of solid tumors, including breast cancer (Kar et al., Genes (Basel), 10(12):961, 2019; Du et al., Stem Cells., 37(7):865-875, 2019; and Jeong et al., Cancer Res., 81(1):77-90, 2021).

Triple negative breast cancer (TNBC) accounts for 15-20% of all breast cancers and mainly affects pre-menopausal women. Although TNBC typically responds initially to standard of care chemotherapy, tumor recurrence commonly occurs within 1 to 3 years post-chemotherapy and is associated with the emergence of organ metastasis and a high incidence of mortality (Chiorean et al., Breast, 22(6):1026-33, 2013). Cancer and tumor cell plasticity promotes high self-renewal capacity, intrinsic resistance to chemotherapeutic agents, and immune evasion capacity, and thus represents one of the difficulties in eradicating TNBC metastasis (Yagata et al., Breast Cancer, 18(3):165-173, 2011; Ascolani et al., PLoS Comput Biol., 11(5):e1004199, 2015; Opyrchal et al., Int J Oncol., 45(3):1193-1199, 2014; and Samanta et al., Proc Natl Acad Sci USA., 115(6):E1239-E1248, 2018).

### SUMMARY

This document provides methods and materials for treating mammals having chemoresistant cancer. For example, methods provided herein can be used to treat mammals having a chemoresistant cancer by administering a NOTCH3 inhibitor and, either simultaneously or subsequently, chemotherapy. In some cases, the methods provided herein can be used to monitor treatment of a mammal having chemoresistant cancer, but measuring the level of NOTCH3 in the cancer after treatment with the NOTCH3 inhibitor. This document also provides methods and materials for identifying mammals with chemoresistant cancer as being likely to respond to treatment with a NOTCH3 inhibitor and chemotherapy. For example, methods provided herein can be used to determine that a mammal has a chemoresistant cancer containing NOTCH3+ cancer cells or NOTCH3-overexpressing cancer cells, and, based on that determination, identifying the mammal as being likely to respond to treatment with a NOTCH3 inhibitor and chemotherapy.

As demonstrated herein, NOTCH3 mRNA expression is an indicator of poor prognosis in TNBC, as high NOTCH3 mRNA expression was significantly linked to reduced recurrence-free survival, indicating that NOTCH3 expression may drive TNBC progression. Also as demonstrated herein, three-dimensional mammospheres established from patient-derived xenografts expressed high levels of NOTCH3 and exhibited high levels of aldehyde dehydrogenase (ALDH) activity, but NOTCH3 genetic targeting reduced ALDH and enhanced chemosensitivity. In addition, a humanized anti-NOTCH3 antibody selectively targeted NOTCH3, induced apoptosis, impaired ALDH activity, and enhanced chemosensitivity in TNBC cells; the antibody also inhibited the growth of highly metastatic TNBC xenografts, reduced intra-tumoral PD-L1 expression, and impaired the immune evasion capacity of TNBC cells. The results presented herein indicate that NOTCH3 inhibition can inhibit the immune evasion capacity of TNBC cells, providing a path to treat chemoresistant cancers. Having the ability to treat mammals (e.g., humans) with chemoresistant cancers provides a unique and unrealized opportunity to overcome chemoresistance and restore or enhance responsiveness to standard of care chemotherapy.

In general, one aspect of this document features a method for treating a mammal having a cancer identified as being resistant to a chemotherapeutic agent. The method can include, or consist essentially of, administering a NOTCH3 inhibitor to the mammal, thereby increasing the susceptibility of the cancer to the chemotherapeutic agent; and administering the chemotherapeutic agent to the mammal. The cancer can be a metastatic cancer. The cancer can contain NOTCH3⁺ cells. The method can further include, prior to administering the NOTCH3 inhibitor, detecting the presence of the NOTCH3⁺ cells. The cancer can contain NOTCH3 over-expressing cells. The method can further include, prior to administering the NOTCH3 inhibitor, detecting the presence of the NOTCH3 over-expressing cells. The cancer can contain ALDH⁺ cells. The method can further include, prior to administering the NOTCH3 inhibitor, detecting the presence of the ALDH⁺ cells.

The cancer can contain ALDH over-expressing cells. The method can further include, prior to administering the NOTCH3 inhibitor, detecting the presence of the ALDH over-expressing cells. The cancer can contain Epithelial to Mesenchymal Transition⁺ (EMT⁺) cells. The method can further include, prior to administering the NOTCH3 inhibitor, detecting the presence of the EMT⁺ cells. The cancer can contain cancer stem-like cells. The method can further include, prior to administering the NOTCH3 inhibitor, detecting the presence of the cancer stem-like cells. The cancer can be TNBC, ovarian cancer, or another solid tumor with elevated NOTCH3 expression. The cancer can be NOTCH3⁺ TNBC, NOTCH3⁺ ovarian cancer, ALDH⁺ TNBC, ALDH⁺ ovarian cancer, ALDH⁺ and NOTCH3⁺ TNBC, or ALDH and NOTCH3⁺ ovarian cancer. The NOTCH3 inhibitor can include a shRNA targeted to NOTCH3 (e.g., a shRNA having the nucleotide sequence set forth in SEQ ID NO:1, SEQ ID NO:2, SEQ ID NO:3, SEQ ID NO:4, or SEQ ID NO:5). The NOTCH3 inhibitor can be antibody (e.g., AV-353). The mammal can have been treated with the chemotherapeutic agent without a complete response more than 4 months prior to administering the NOTCH3 inhibitor. The method can include administering the NOTCH3 inhibitor no more than 4 months prior to administering the chemotherapeutic agent. The method can include administering the chemotherapeutic agent no more than 4 months prior to administering the NOTCH3 inhibitor. The method can include administering the NOTCH3 inhibitor with the chemotherapeutic agent. The chemotherapeutic agent can be selected from the group consisting of paclitaxel, cabazitaxel, docetaxel, Nab-paclitaxel, vinblastine, vincristine, vinorelbine, altretamine, bendamustine, busulfan, carboplatin, carmustine, chlorambucil, cisplatin, cyclophosphamide, dacarbazine, ifosfamide, lomustine, mechlorethamine, melphalan, oxaliplatin, temozolomide, thiotepa, trabectedin, azacitidine, 5-fluorouracil, 6-mercaptopurine, capecitabine, cladribine, clofarabine, cytarabine, decitabine, floxuridine, fludarabine, gemcitabine, hydroxyurea, methotrexate, nelarabine, pemetrexed, pentostatin, pralatrexate, thioguanine, dunorubicin, doxorubicin, epirubicin, idarubicin, valrubicin, bleomycin, dactinomycin, mitomycin-C, mitoxantrone, irinotecan, topotecan, etoposide, and teniposide. The method can further include, after administering the NOTCH3 inhibitor, monitoring NOTCH3 levels in the mammal, wherein a decrease in the NOTCH3 levels indicates successful treatment with the NOTCH3 inhibitor. The method can further include, after administering the NOTCH3 inhibitor, monitoring PD-L1 levels in the mammal, where a decrease in the PD-L1 levels indicates successful treatment with the NOTCH3 inhibitor. The method can further include administering to the mammal a checkpoint inhibitor (e.g., nivolumab, pembrolizumab, cemiplimab, ipilimumab, tremelimumab, atezolizumab, avelumab, or durvalumab).

In another aspect, this document features method for identifying a mammal as having a cancer resistant to a chemotherapeutic agent and as being likely to respond to treatment with a NOTCH3 inhibitor and the chemotherapeutic agent. The method can include, or consist essentially of, measuring a level of NOTCH3 in cells from the cancer, and when the measured level of NOTCH3 is elevated as compared to a control level of NOTCH3 in normal tissue, identifying the mammal as being likely to respond to treatment with the NOTCH3 inhibitor and the chemotherapeutic agent as opposed to treatment with the chemotherapeutic agent in the absence of the NOTCH3 inhibitor. The cancer can be a metastatic cancer. The cancer can be TNBC, ovarian cancer, or another solid tumor with elevated NOTCH expression. The mammal can have been treated with the chemotherapeutic agent without a complete response more than 4 months prior to administering the NOTCH3 inhibitor. The method can further include administering the NOTCH3 inhibitor and the chemotherapeutic agent to the mammal. The method can include administering the NOTCH3 inhibitor no more than 4 months prior to administering the chemotherapeutic agent. The method can include administering the chemotherapeutic agent no more than 4 months prior to administering the NOTCH3 inhibitor. The method can include administering the NOTCH3 inhibitor with the chemotherapeutic agent. The chemotherapeutic agent can be selected from the group consisting of paclitaxel, cabazitaxel, docetaxel, Nab-paclitaxel, vinblastine, vincristine, vinorelbine, altretamine, bendamustine, busulfan, carboplatin, carmustine, chlorambucil, cisplatin, cyclophosphamide, dacarbazine, ifosfamide, lomustine, mechlorethamine, melphalan, oxaliplatin, temozolomide, thiotepa, trabectedin, azacitidine, 5-fluorouracil, 6-mercaptopurine, capecitabine, cladribine, clofarabine, cytarabine, decitabine, floxuridine, fludarabine, gemcitabine, hydroxyurea, methotrexate, nelarabine, pemetrexed, pentostatin, pralatrexate, thioguanine, dunorubicin, doxorubicin, epirubicin, idarubicin, valrubicin, bleomycin, dactinomycin, mitomycin-C, mitoxantrone, irinotecan, topotecan, etoposide, and teniposide. The method can further include, after administering the NOTCH3 inhibitor, monitoring NOTCH3 levels in the mammal, where a decrease in the NOTCH3 levels indicates successful treatment with the NOTCH3 inhibitor.

In another aspect, this document features a method for identifying a mammal as having a cancer resistant to a chemotherapeutic agent and as being likely to respond to treatment with a NOTCH3 inhibitor and the chemotherapeutic agent, where the method includes, or consists essentially of, measuring a level of ALDH activity in cells from the cancer, and when the measured level of ALDH activity is elevated as compared to a control level of ALDH, identifying the mammal as being likely to respond to treatment with the NOTCH3 inhibitor and the chemotherapeutic agent as opposed to treatment with the chemotherapeutic agent in the absence of the NOTCH3 inhibitor. The cancer can be a metastatic cancer. The cancer can be TNBC, ovarian cancer, or another solid tumor with elevated NOTCH3 expression. The mammal can have been treated with the chemotherapeutic agent without a complete response more than 4 months prior to administering the NOTCH3 inhibitor. The method can further include administering the NOTCH3 inhibitor and the chemotherapeutic agent to the mammal. The method can include administering the NOTCH3 inhibitor no more than 4 months prior to administering the chemotherapeutic agent. The method can include administering the chemotherapeutic agent no more than 4 months prior to administering the NOTCH3 inhibitor. The method can include administering the NOTCH3 inhibitor with the chemotherapeutic agent. The chemotherapeutic agent can be selected from the group consisting of paclitaxel, cabazitaxel, docetaxel, Nab-paclitaxel, vinblastine, vincristine, vinorelbine, altretamine, bendamustine, busulfan, carboplatin, carmustine, chlorambucil, cisplatin, cyclophosphamide, dacarbazine, ifosfamide, lomustine, mechlorethamine, melphalan, oxaliplatin, temozolomide, thiotepa, trabectedin, azacitidine, 5-fluorouracil, 6-mercaptopurine, capecitabine, cladribine, clofarabine, cytarabine, decitabine, floxuridine, fludarabine, gemcitabine, hydroxyurea, methotrexate, nelarabine, pemetrexed, pentostatin, pralatrexate, thioguanine, dunorubicin, doxorubicin, epirubicin, idarubicin, valrubicin, bleomycin, dactinomycin, mitomycin-C, mitoxantrone, irinotecan, topotecan, etoposide, and teniposide. The method can further include, after administering the NOTCH3 inhibitor, monitoring NOTCH3 levels in the mammal, where a decrease in the NOTCH3 levels indicates successful treatment with the NOTCH3 inhibitor.

In still another aspect, this document features a method for monitoring treatment of a mammal having a cancer resistant to a chemotherapeutic agent. The method can include, or consist essentially of, identifying the mammal as having an elevated level of NOTCH3 in cells from the cancer, administering a NOTCH3 inhibitor to the mammal, and measuring a post-treatment level of NOTCH3 in cells of the cancer after the administering, where a decrease in the post-treatment level of NOTCH3 as compared to the elevated level of NOTCH3 indicates successful treatment of the mammal. The cancer can be a metastatic cancer. The cancer can be TNBC, ovarian cancer, or another solid tumor with elevated NOTCH3 expression. The cancer can be NOTCH3⁺ TNBC, NOTCH3⁺ ovarian cancer, ALDH⁺ TNBC, ALDH⁺ ovarian cancer, ALDH⁺ and NOTCH3⁺ TNBC, or ALDH and NOTCH3⁺ ovarian cancer. The NOTCH3 inhibitor can include a shRNA targeted to NOTCH3 (e.g., a shRNA having the nucleotide sequence set forth in SEQ ID NO:1, SEQ ID NO:2, SEQ ID NO: 3, SEQ ID NO:4, or SEQ ID NO:5). The NOTCH3 inhibitor can include an antibody (e.g., a humanized antibody such as AV-353). The mammal can have been treated with the chemotherapeutic agent without a complete response more than 4 months prior to administering the NOTCH3 inhibitor. The method can further include administering the chemotherapeutic agent to the mammal. The method can include administering the NOTCH3 inhibitor no more than 4 months prior to administering the chemotherapeutic agent. The method can include administering the chemotherapeutic agent no more than 4 months prior to administering the NOTCH3 inhibitor. The method can include administering the NOTCH3 inhibitor with the chemotherapeutic agent. The chemotherapeutic agent can be selected from the group consisting of paclitaxel, cabazitaxel, docetaxel, Nab-paclitaxel, vinblastine, vincristine, vinorelbine, altretamine, bendamustine, busulfan, carboplatin, carmustine, chlorambucil, cisplatin, cyclophosphamide, dacarbazine, ifosfamide, lomustine, mechlorethamine, melphalan, oxaliplatin, temozolomide, thiotepa, trabectedin, azacitidine, 5-fluorouracil, 6-mercaptopurine, capecitabine, cladribine, clofarabine, cytarabine, decitabine, floxuridine, fludarabine, gemcitabine, hydroxyurea, methotrexate, nelarabine, pemetrexed, pentostatin, pralatrexate, thioguanine, dunorubicin, doxorubicin, epirubicin, idarubicin, valrubicin, bleomycin, dactinomycin, mitomycin-C, mitoxantrone, irinotecan, topotecan, etoposide, and teniposide.

In another aspect, this document features a method for treating a mammal having a cancer identified as being resistant to a chemotherapeutic agent, where the method includes, or consists essentially of, administering a NOTCH3 inhibitor to the mammal, thereby increasing the susceptibility of the cancer to the chemotherapeutic agent. The cancer can be a metastatic cancer. The cancer can contain NOTCH3+ cells. The method can further include, prior to administering the NOTCH3 inhibitor, detecting the presence of the NOTCH3+ cells. The cancer can contain NOTCH3 over-expressing cells. The method can further include, prior to administering the NOTCH3 inhibitor, detecting the presence of the NOTCH3 over-expressing cells. The cancer can contain ALDH+ cells. The method can further include, prior to administering the NOTCH3 inhibitor, detecting the presence of the ALDH+ cells. The cancer can contain ALDH over-expressing cells. The method can further include, prior to administering the NOTCH3 inhibitor, detecting the presence of the ALDH over-expressing cells. The cancer can contain EMT+ cells. The method can further include, prior to administering the NOTCH3 inhibitor, detecting the presence of the EMT+ cells. The cancer can contain cancer stem-like cells. The method can further include, prior to administering the NOTCH3 inhibitor, detecting the presence of the cancer stem-like cells. The cancer can be TNBC, ovarian cancer, or another solid tumor with elevated NOTCH3 expression. The cancer can be NOTCH3+ TNBC, NOTCH3+ ovarian cancer, ALDH+ TNBC, ALDH+ ovarian cancer, ALDH+ and NOTCH3+ TNBC, or ALDH+ and NOTCH3+ ovarian cancer. The NOTCH3 inhibitor can include a shRNA targeted to NOTCH3 (e.g., a shRNA having the nucleotide sequence set forth in SEQ ID NO:1, SEQ ID NO:2, SEQ ID NO:3, SEQ ID NO:4, or SEQ ID NO:5). The NOTCH3 inhibitor can be antibody (e.g., a humanized antibody such as AV-353). The mammal can have been treated with the chemotherapeutic agent without a complete response more than 4 months prior to administering the NOTCH3 inhibitor. The method can further include, after administering the NOTCH3 inhibitor, monitoring NOTCH3 levels in the mammal, wherein a decrease in the NOTCH3 levels indicates successful treatment with the NOTCH3 inhibitor. The method can further include, after administering the NOTCH3 inhibitor, monitoring PD-L1 levels in the mammal, where a decrease in the PD-L1 levels indicates successful treatment with the NOTCH3 inhibitor. The method can further include administering to the mammal a checkpoint inhibitor (e.g., nivolumab, pembrolizumab, cemiplimab, ipilimumab, tremelimumab, atezolizumab, avelumab, or durvalumab).

In another aspect, this document features the use of a NOTCH3 inhibitor and a chemotherapeutic agent for treating a mammal having a cancer identified as being resistant to the chemotherapeutic agent, wherein administration of the NOTCH3 inhibitor to the mammal increases the susceptibility of the cancer to the chemotherapeutic agent. The cancer can be a metastatic cancer. The cancer can include NOTCH3⁺ cells. The cancer can include ALDH⁺ cells. The cancer can include Epithelial to Mesenchymal Transition⁺ (EMT⁺) cells. The cancer can include cancer stem-like cells. The cancer can be TNBC, ovarian cancer, or another solid tumor with elevated NOTCH3 expression. The use can further include a checkpoint inhibitor for treating the mammal.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIG. 1 is a set of four Kaplan-Meier plots showing the correlation between NOTCH mRNA expression (upper left—NOTCH1, upper right—NOTCH2, bottom left—NOTCH3, and bottom right—NOTCH4) and Recurrence-Free Survival (RFS) in a cohort of 107 lymph-node+ TNBC patients.

FIGS. 2A and 2B show NOTCH3 expression and ALDH activity in unique TNBC models. FIG. 2A is an immunoblot assay showing expression of NOTCH receptors in TNBC 3DMammospheres (MPS). FIG. 2B shows ALDH activity measured by FACS on 10,000 cells isolated from 3D-MPS using the ALDEFLUOR™ assay. Samples treated with DEAB were used as negative control. The graph indicates the number of ALDH+ cells. Three independent experiments were performed (±S.D.).

FIGS. 3A and 3B showNOTCH3 genetic targeting in TNBC-M40 MPS. FIG. 3A is a pair of images from immunofluorescence analysis, showing NOTCH3 expression (red) in TNBCM40 MPS infected with Scrambled Lenti-shRNAs (control) or NOTCH3 Lenti-shRNAs. Nuclei were stained with 4′,6-diamidino-2-phenylindole (DAPI) (blue). The red (NOTCH3) staining was greatly reduced with the NOTCH3 Lenti-shRNAs. FIG. 3B shows ALDH activity measured by FACS analysis (left) on 10,000 events using the ALDEFLUOR™ assay kit. The graph (right) plots the number of ALDH+ cells. Three independent experiments were performed in triplicate (±S.D.).

FIGS. 4A and 4B show NOTCH3 genetic targeting in TNBC-M25 mammospheres. FIG. 4A includes images of TNBCM25 single-cell dilutions grown under non-adherent conditions. After 48 hours of incubation, mammospheres were treated with 5 nM (½ IC50) or 10 nM (IC50) docetaxel (DTX) for 10 days. FIG. 4B is a graph plotting mammosphere area measured using the NIH Image-J software. Three independent experiments were performed in triplicate (±S.D.).

FIG. 5 shows the result of NOTCH3 pharmacologic targeting in SUM149-PT TNBC cells, with an immunoblot analysis of expression of NOTCH-intracellular domain (ICD) proteins before and after treatment with AV-353 (IC50: 200 ng).

FIGS. 6A and 6B show results from NOTCH3 pharmacologic targeting in SUM149-PT TNBC cells. FIG. 6A is a graph plotting the results of a real-time apoptosis assay (INCUCYTE®) before and after AV-353 (IC50: 200 ng) treatment. Three independent experiments were performed (±S.D.). FIG. 6B is a graph plotting ALDH activity as measured by FACS on 10,000 events using the ALDEFLUOR™ assay before and after AV-353 (IC50: 200 ng) treatment. Samples treated with DEAB were used as a negative control. The graph plots the number of ALDH+ cells.

FIGS. 7A and 7B show results from treatment of 3D-mammospheres (3D-MPS) of SM149-PT TNBC cells with AV-353 (½ IC50: 100 ng) and/or DTX (½ IC50: 5 nM). FIG. 7A includes representative images of RFP-tagged 3DMPS before and after treatment with AV-353 (½ IC50: 100 ng) and/or DTX (½ IC50: 5 nM) for 96 hours, using Annexin-V (red) as a marker of early apoptosis. FIG. 7B is a graph plotting real-time growth of RFP-tagged 3D-MPS before and after treatment with AV-353 (½ IC50: 100 ng) and/or DTX (½ IC50: 5 nM) for 96 hours. 3D-MPS growth was quantified using the INCUCYTE® Cell Player System. Three independent experiments were performed in triplicate (±S.D.).

FIGS. 8A-8C show the effects of AV-353 on TNBC xenograft growth and tumor infiltration of CD8+ T-cells. FIG. 8A is a graph plotting tumor volume. 1×10⁶ of MDA-MB 231/LM cells were transplanted into the 4th mammary fat pad (MFP) of female NSG-CD34+ humanized mice (5 animals per group). When the tumor size reached 50 mm³, mice were treated with AV-353 (20 mg/Kg, IP injections, three times per week). Tumor size was measured with a digital caliper. Control Vs AV-353: P<0.05. FIG. 8B is a graph plotting body weight in grams. Body weight of animals randomized in the placebo (control) and AV-353-treated groups was monitored during the preclinical trial.

FIG. 8C includes a pair of images from immunofluorescence analyses showing expression of CD8 (arrows) in MDA-MB 231/LM xenografts without (“control”) and with AV-353 Ab treatment. Nuclei were stained with DAPI.

FIGS. 9A and 9B show intra-tumoral PD-L1 expression in TNBC cells. FIG. 9A includes an image showing an immunoblot of PD-L1 expression in MDA-MB 231 and MD-MB 231 LM cells with densitometric analysis plotted in the graph below. FIG. 9B includes an image of an immunoblot showing PD-L1 expression in MDA-MB 231 LM TNBC cells before and after treatment with AV-353 (IC50: 200 ng) for 48 hours. Densitometric analysis showing the fold change of PD-L1 protein levels normalized to α-tubulin was performed using ImageJ-NIH Software, and is plotted in the graph below the image.

FIG. 10 is a graph plotting tumor size after NOTCH3 pharmacologic targeting. 4T1 syngeneic TNBC tumor cells (1×10⁶) were transplanted into the 4th mammary fat pad (MFP) of BALB/c female mice (5 animals per group). When the tumor size reached 50 mm³, mice were treated with 28042 Ab (20 mg/Kg by intraperitoneal (i.p.) injection, three times per week). Tumor size was measured with a digital caliper.

FIG. 11 includes a series of images showing NOTCH3, PD-L1, and CD8 expression in 4T1 syngeneic tumor tissues. Immunofluorescence analysis showed expression of NOTCH3, PD-L1, and CD8 in tumor tissues before and after treatment of 4T1 syngeneic tumors with 28042 Ab (see, FIG. 10). Nuclei were stained with DAPI.

FIG. 12A is a graph plotting green area confluence overtime of GFP-tagged MDA-MB 231 LM cells, with and without treatment with AV-353 (IC50: 200 ng). FIG. 12B is a graph plotting red count per image (Red Annexin-V) over time, with and without treatment with AV-353 (IC50: 200 ng).

FIG. 13A includes an image showing an immunoblot (top) and a graph plotting densitometric analysis of PD-L1 in MDA-MB 231 LM cells treated with scrambled, lenti-shRNA(A) or PD-L1(B) lenti-shRNA. FIG. 13B is a graph plotting the results of an ALDEFLUOR™ assay of 10,000 events in MDA-MD 231 LM cells treated with scrambled shRNA or shRNAPD-L1(A).

FIG. 14A is an image indicating tumor size resulting from MDA-MB 231 LM cells implanted in mice at day 0 and day 7 when treated with scrambled or PD-L1 shRNA. FIG. 14B is a pair of graphs plotting tumor growth area.

FIG. 15 is a graph plotting body weight of NSG female mice treated with saline (CON), paclitaxel 10 mg/Kg (PTX), paclitaxel 10 mg/Kg+AV-353 100 ug (PTX+AV100), paclitaxel 10 mg/Kg+AV-353 200 ug (PTX+AV200), or paclitaxel 10 mg/Kg+AV-353 400 ug (PTX+AV400) for four weeks.

FIG. 16 is a set of histology images of mouse liver and stomach after various treatments with PTX and/or AV-353. These images show that there were no significant changes in the architecture of liver and stomach tissue with either agent along or in combination. The lack of significant damage to the liver and stomach tissues suggested that PTX and AV-353 are safe to use in these tissues.

FIG. 17 is a series of graphs plotting liver toxicity in mice treated with saline (CON), paclitaxel 10 mg/Kg (PTX), paclitaxel 10 mg/Kg+AV-353 100 ug (PTX+AV100), paclitaxel 10 mg/Kg+AV-353 200 ug (PTX+AV200), or paclitaxel 10 mg/Kg+AV-353 400 ug (PTX+AV400), based on measurements of liver function indicators: alanine aminotransferase (ALT), aspartate aminotransferase (AST), alkaline phosphatase (ALP), and total bilirubin (TBIL), as well as an indicator of kidney function (creatinine; CRE), and an indicator of general nutritional status (albumin; ALB). Blood samples (1 ml) were collected from animals, and liver enzyme analysis was carried out using a VETSCAN® instrument (Zoetis; Parsippany, NJ).

FIG. 18A is a set of four Kaplan-Meier plots showing the correlation between NOTCH mRNA expression (upper left—NOTCH1, upper right—NOTCH2, bottom left—NOTCH3, and bottom right—NOTCH4) and Recurrence-Free Survival (RFS) in a cohort of 107 lymph-node+ TNBC patients. FIG. 18B is a series of graphs plotting the prognostic value of NOTCH gene expression using the five-year distant disease-free survival (DDFS) data from both the Mayo Clinic RNA-Seq cohort and the claudin-low cohort. Among the four NOTCH genes, higher expression of NOTCH2 and NOTCH3 genes was marginally associated with worse DDFS outcomes for all TNBC subtypes, while higher expression of NOTCH3 was associated with poor DDFS outcomes in claudin-low patients.

FIGS. 19A and 19B show NOTCH3 expression and ALDH activity in unique TNBC models. FIG. 19A is an immunoblot assay showing expression of NOTCH receptors in TNBC 3DMammospheres (MPS). FIG. 19B shows ALDH activity measured by FACS on 10,000 cells isolated from 3D-MPS using the ALDEFLUOR™ assay. Samples treated with DEAB were used as negative control. The graph indicates the number of ALDH+ cells. Three independent experiments were performed (±S.D.). FIGS. 19C and 19D showNOTCH3 genetic targeting in TNBC-M40 MPS. FIG. 19C is a pair of images from immunofluorescence analysis, showing NOTCH3 expression (red) in TNBCM40 MPS infected with Scrambled Lenti-shRNAs (control) or NOTCH3 Lenti-shRNAs. Nuclei were stained with 4′,6-diamidino-2-phenylindole (DAPI) (blue). The red (NOTCH3) staining was greatly reduced with the NOTCH3 Lenti-shRNAs. FIG. 19D shows ALDH activity measured by FACS analysis (left) on 10,000 events using the ALDEFLUOR™ assay kit. The graph (right) plots the number of ALDH+ cells. Three independent experiments were performed in triplicate (±S.D.). FIG. 19E shows the result of NOTCH3 pharmacologic targeting in SUM149-PT TNBC cells, with an immunoblot analysis of expression of NOTCH-intracellular domain (ICD) proteins before and after treatment with AV-353 (IC50: 200 ng). FIGS. 19F and 19G show results from NOTCH3 pharmacologic targeting in SUM149-PT TNBC cells. FIG. 19F is a graph plotting ALDH activity as measured by FACS on 10,000 events using the ALDEFLUOR™ assay before and after AV-353 (IC50: 200 ng) treatment. Samples treated with DEAB were used as a negative control. The graph plots the number of ALDH+ cells. FIG. 19G is a graph plotting the results of a real-time apoptosis assay (INCUCYTE®) before and after AV-353 (IC50: 200 ng) treatment. Three independent experiments were performed (±S.D.).

FIGS. 20A and 20B show NOTCH3 genetic targeting in TNBC-M25 mammospheres. FIG. 20A includes images of TNBCM25 single-cell dilutions grown under non-adherent conditions. After 48 hours of incubation, mammospheres were treated with 5 nM (½ IC50) or 10 nM (IC50) docetaxel (DTX) for 10 days. FIG. 20B is a graph plotting mammosphere area measured using the NIH Image-J software. Three independent experiments were performed in triplicate (±S.D.). FIGS. 20C and 20D show results from treatment of 3D-mammospheres (3D-MPS) of SM149-PT TNBC cells with AV-353 (½ IC50: 100 ng) and/or DTX (½ IC50: 5 nM). FIG. 20C includes representative images of RFP-tagged 3DMPS before and after treatment with AV-353 (½ IC50: 100 ng) and/or DTX (½ IC50: 5 nM) for 96 hours, using Annexin-V (red) as a marker of early apoptosis. FIG. 20D is a graph plotting real-time growth of RFP-tagged 3D-MPS before and after treatment with AV-353 (½ IC50: 100 ng) and/or DTX (½ IC50: 5 nM) for 96 hours. 3D-MPS growth was quantified using the INCUCYTE® Cell Player System. Three independent experiments were performed in triplicate (±S.D.).

FIG. 21A is a graph plotting green area confluence overtime of GFP-tagged MDA-MB 231 LM cells, with and without treatment with AV-353 (IC50: 200 ng). FIG. 21B is a graph plotting red count per image (Red Annexin-V) over time, with and without treatment with AV-353 (IC50: 200 ng).

FIG. 22A is a graph plotting body weight of NSG female mice treated with saline (CON), paclitaxel 10 mg/Kg (PTX), paclitaxel 10 mg/Kg+AV-353 100 ug (PTX+AV100), paclitaxel 10 mg/Kg+AV-353 200 ug (PTX+AV200), or paclitaxel 10 mg/Kg+AV-353 400 ug (PTX+AV400) for four weeks. FIG. 22B is a set of histology images of mouse liver and stomach after various treatments with PTX and/or AV-353. These images show that there were no significant changes in the architecture of liver and stomach tissue with either agent along or in combination. The lack of significant damage to the liver and stomach tissues suggested that PTX and AV-353 are safe to use in these tissues. FIG. 22C is a series of graphs plotting liver toxicity in mice treated with saline (CON), paclitaxel 10 mg/Kg (PTX), paclitaxel 10 mg/Kg+AV-353 100 ug (PTX+AV100), paclitaxel 10 mg/Kg+AV-353 200 ug (PTX+AV200), or paclitaxel 10 mg/Kg+AV-353 400 ug (PTX+AV400), based on measurements of liver function indicators: alanine aminotransferase (ALT), aspartate aminotransferase (AST), alkaline phosphatase (ALP), and total bilirubin (TBIL), as well as an indicator of kidney function (creatinine; CRE), and an indicator of general nutritional status (albumin; ALB). Blood samples (1 ml) were collected from animals, and liver enzyme analysis was carried out using a VETSCAN® instrument (Zoetis; Parsippany, NJ).

FIGS. 23A-23D show the effects of AV-353 on TNBC xenograft growth and tumor infiltration of CD8+ T-cells. FIG. 23A is a graph plotting tumor volume. 1×10⁶ of MDA-MB 231/LM cells were transplanted into the 4th mammary fat pad (MFP) of female NSG-CD34+ humanized mice (5 animals per group). When the tumor size reached 50 mm³, mice were treated with AV-353 (20 mg/Kg, IP injections, three times per week). Tumor size was measured with a digital caliper. Control Vs AV-353: P<0.05.

FIG. 23B is a graph plotting tumor size after NOTCH3 pharmacologic targeting. 4T1 syngeneic TNBC tumor cells (1×10⁶) were transplanted into the 4th mammary fat pad (MFP) of BALB/c female mice (5 animals per group). When the tumor size reached 50 mm³, mice were treated with 28042 Ab (20 mg/Kg by intraperitoneal (i.p.) injection, three times per week). Tumor size was measured with a digital caliper. FIGS. 23C and 23D include images from two mouse models treated with NOTCH3 blocking antibodies, showing CD8 expression in 4T1 syngeneic tumor tissues. Immunofluorescence analysis showed expression of CD8 in tumor tissues before and after treatment of 4T1 syngeneic tumors with 28042 Ab. Nuclei were stained with DAPI.

FIG. 24 includes a series of images showing NOTCH3 and PD-L1 expression in 4T1 syngeneic tumor tissues. Immunofluorescence analysis showed expression of NOTCH3 and PD-L1 in tumor tissues before and after treatment of 4T1 syngeneic tumors with 28042 Ab. Nuclei were stained with DAPI.

FIG. 25 includes an image showing an immunoblot of PD-L1 expression in MDA-MB 231 and MD-MB 231 LM cells, with densitometric analysis plotted in the graph below.

FIG. 26A is an image of a Western blot and FIG. 26B is a graph showing that knockdown of NOTCH3 protein levels in MDA-MB-231/LM cells led to a reduction in expression of the PD-L1 ligand. FIG. 26C is an image of an immunoblot showing PD-L1 expression in MDA-MB 231 LM TNBC cells before and after treatment with AV-353 (IC50: 200 ng) for 48 hours. Densitometric analysis showing the fold change of PD-L1 protein levels normalized to α-tubulin was performed using ImageJ-NIH Software, and is plotted in the graph (FIG. 26D) below the image.

FIGS. 27A-27C show that NOTCH3 acts upstream over PD-L1 and that a substantial fraction of genes involved in EMT-mediated cancer cell plasticity and stemness regulated by NOTCH3 are also regulated by PD-L1. FIG. 27A is a Venn diagram from a transcriptomic analysis performed using MDA-MB-231/LM cells in which NOTCH3 or PD-L1 was stably knocked down by shRNA. In total, 1742 DEGs were identified from the PD-L1 vs control set and 1716 DEGs were identified from the NOTCH3 versus the control set. More than half of the genes (953 DEGs) were common between the two datasets. FIG. 27B shows that among those 953 DEGs, genes were identified that regulate EMT, immune evasion, sternness and metastasis. FIG. 27C is a heatmap showing that NOTCH3 or PD-L1 genetic targeting induced a significant down-regulation of EMT and stemness genes.

FIG. 28A includes an image showing an immunoblot (top) and a graph plotting densitometric analysis of PD-L1 in MDA-MB 231 LM cells treated with scrambled, lenti-shRNA(A) or PD-L1(B) lenti-shRNA. FIG. 28B is a graph plotting the results of an ALDEFLUOR™ assay of 10,000 events in MDA-MD 231 LM cells treated with scrambled shRNA or shRNA PD-L1(A). FIG. 28C is an image indicating tumor size resulting from MDA-MB 231 LM cells implanted in mice at day 0 and day 7 when treated with scrambled or PD-L1 shRNA. FIG. 28D is a pair of graphs plotting tumor growth area.

### DETAILED DESCRIPTION

This document provides methods and materials for identifying and/or treating mammals having a cancer that is resistant to treatment with one or more chemotherapeutic agents. For example, this document provides methods and materials for identifying a mammal (e.g., a human) having a chemotherapy-resistant cancer (e.g., breast cancer or ovarian cancer that is resistant to treatment with a chemotherapeutic agent) as having an elevated level of NOTCH3, an elevated level of ALDH (e.g., ALDH1) activity, an elevated level of ALDH (e.g., ALDH1) expression, or any combination thereof, in chemotherapy-resistant cancer cells. This document also provides methods and materials for administering one or more NOTCH3 inhibitors, with or without one or more chemotherapeutic agents, to a mammal having a chemotherapy-resistant cancer identified as having an elevated level of NOTCH3 and/or elevated ALDH activity and/or elevated ALDH expression. In some cases, for example, this document provides methods and materials for administering one or more NOTCH3 inhibitors to a mammal having a chemotherapy-resistant cancer identified as having an elevated level of NOTCH3 and/or elevated ALDH activity and/or elevated ALDH expression, such that cancer becomes susceptible to chemotherapy, and administering—at the same time as or after the one or more NOTCH3 inhibitors—one or more chemotherapeutic agents.

Cancers that can be treated with the methods and materials provided herein are, in general, chemoresistant (e.g., chemoresistant breast cancers and chemoresistant ovarian cancers). In some cases, a chemoresistant cancer that can be treated according to the methods provided herein is a metastatic cancer (e.g., a metastatic breast cancer or a metastatic ovarian cancer). In some cases, a chemoresistant cancer that can be treated according to the methods provided herein can contain NOTCH3 positive cells (e.g., cells that express NOTCH3), and/or NOTCH3 over-expressing cells (e.g., cells that express NOTCH3 at an elevated level). In some cases, a chemoresistant cancer that can be treated according to the methods provided herein can contain ALDH positive cells (e.g., cells that express ALDH), ALDH over-expressing cells (e.g., cells that express ALDH at an elevated level), and/or cells that have an elevated level of ALDH activity. In some cases, a chemoresistant cancer that can be treated according to the methods provided herein can contain EMT positive cells (e.g., cells that express EMT), EMT over-expressing cells (e.g., cells that express EMT at an elevated level), and/or cells that exhibit plasticity or tumor sternness reprogramming. In some cases, a chemoresistant cancer that can be treated according to the methods provided herein is TNBC. In some cases, a chemoresistant cancer that can be treated according to the methods described herein can be, without limitation, a NOTCH3 positive TNBC, NOTCH3 positive ovarian cancer, ALDH positive TNBC, ALDH positive ovarian cancer, TNBC with elevated ALDH activity, ovarian cancer with elevated ALDH activity, NOTCH3 positive and ALDH positive TNBC, NOTCH3 positive and ALDH positive ovarian cancer, NOTCH3 positive TNBC with elevated ALDH activity, NOTCH3 positive ovarian cancer with elevated ALDH activity, NOTCH3 over-expressing TNBC, NOTCH3 over-expressing ovarian cancer, ALDH over-expressing TNBC, ALDH over-expressing ovarian cancer, NOTCH3 over-expressing and ALDH over-expressing TNBC, NOTCH3 over-expressing and ALDH over-expressing ovarian cancer, NOTCH3 over-expressing TNBC with elevated ALDH activity, or NOTCH3 over-expressing ovarian cancer with elevated ALDH activity.

Any appropriate mammal having a cancer that is resistant to treatment with one or more chemotherapeutic agents can be identified as having an elevated level of NOTCH3, as having an elevated level of ALDH, and/or as having elevated ALDH activity. For example, humans and other primates such as monkeys having a chemotherapy-resistant cancer can be identified as having an elevated level of NOTCH3 within the chemotherapy-resistant cancer. In some cases, any appropriate mammal having a chemotherapy-resistant cancer can be identified as having an elevated level of ALDH. For example, humans and other primates such as monkeys having a chemotherapy-resistant cancer can be identified as having an elevated level of ALDH within the chemotherapy-resistant cancer. In some cases, any appropriate mammal having a chemotherapy-resistant cancer can be identified as having an elevated level of ALDH activity. For example, humans and other primates such as monkeys having a chemotherapy-resistant cancer can be identified as having an elevated level of ALDH activity within the chemotherapy-resistant cancer. In some cases, any appropriate mammal having a chemotherapy-resistant cancer can be identified as having an elevated level of NOTCH3 and an elevated level of ALDH activity. For example, humans and other primates such as monkeys having a chemotherapy-resistant cancer can be identified as having and elevated level of NOTCH3 and an elevated level of ALDH activity within the chemotherapy-resistant cancer. In some cases, dogs, cats, horses, cows, pigs, sheep, mice, or rats having a chemotherapy-resistant cancer can be identified as having an elevated level of NOTCH3 and/or as having an elevated level of ALDH activity within the chemotherapy-resistant cancer.

A mammal (e.g., a human) having a cancer that is resistant to treatment with a chemotherapeutic agent can, in some cases, have been administered the chemotherapeutic agent prior to being assessed to determine whether the cancer contains cells with an elevated level of NOTCH3, an elevated level of ALDH, and/or an elevated level of ALDH activity. For example, a mammal having cancer can have been treated with a chemotherapeutic agent about 1 to 2 weeks, about 2 to 4 weeks, about 1 to 2 months, about 2 to 3 months, at least 2 months, at least 3 months, or at least 4 months prior to being assessed to determine whether the cancer contains cells with an elevated level of NOTCH3, an elevated level of ALDH, and/or an elevated level of ALDH activity. The lack of response (e.g., a complete response) to administration of the chemotherapeutic agent can serve as an indication that the cancer is resistant to treatment with the chemotherapeutic agent. In some cases, a mammal (e.g., a human) having cancer can be assessed to determine whether the cancer contains cells with an elevated level of NOTCH3, an elevated level of ALDH, and/or an elevated level of ALDH activity without previously having been treated with a chemotherapeutic agent.

Any appropriate method can be used to determine if a mammal (e.g., a human) has cells or tissue (e.g., a breast or ovarian biopsy) having (a) an elevated level of NOTCH3, (b) an elevated level of ALDH, and/or (c) an elevated level of ALDH activity. In some cases, for example, any appropriate method can be used to determine if a mammal (e.g., a human) has chemotherapy-resistant tissue (a) having an elevated level of NOTCH3, and (b) having an elevated level of ALDH activity. For example, methods such as immunohistochemistry (IHC) techniques, immunofluorescence (IF) techniques, mass spectrometry-based proteomics, or Western blot techniques can be used to determine if a mammal (e.g., a human) has tissue (e.g., breast or ovarian tissue) having an elevated level of NOTCH3 and/or an elevated level of ALDH. In some cases, a tissue sample (e.g., a breast biopsy or an ovarian biopsy) obtained from a mammal can be stained using an anti-NOTCH3 antibody to determine if the mammal has tissue having an elevated level of NOTCH3 polypeptide. In some cases, a tissue sample (e.g., a breast biopsy or an ovarian biopsy) obtained from a mammal can be stained using an anti-ALDH antibody to determine if the mammal has tissue having an elevated level of ALDH polypeptide. In some cases, mRNA levels can be used as an indicator of polypeptide levels, and can be used to determine whether a tissue (e.g., breast tissue or ovarian tissue) has an elevated level of NOTCH3 and/or an elevated level of ALDH. Any appropriate method of quantifying mRNA can be used to determine whether a tissue has an elevated level of NOTCH3 and/or an elevated level of ALDH. Examples of methods of quantifying mRNA include, without limitation, qRT-PCR, RNA-sequencing, microfluidic capillary electrophoresis, and in situ hybridization. In some cases, ALDH activity can be measured using an ALDEFLUOR™ assay kit (STEMCELL™ Technologies; Vancouver, BC) or an ALDH Activity Assay Kit (AbCam; Cambridge, United Kingdom).

Any appropriate sample can be used to determine if a mammal (e.g., a human) has tissue (a) having an elevated level of NOTCH3, (b) having an elevated level of ALDH, and/or (c) having an elevated level of ALDH activity. For example, a breast tissue biopsy obtained from a mammal (e.g., a human) can be used to determine if the mammal has breast tissue with an elevated level of NOTCH3, or an ovarian tissue biopsy obtained from a mammal (e.g., a human) can be used to determine if the mammal has ovarian tissue with an elevated level of NOTCH3. In some cases, a breast tissue biopsy obtained from a mammal (e.g., a human) can be used to determine if the mammal has breast tissue with an elevated level of ALDH, or an ovarian tissue biopsy obtained from a mammal (e.g., a human) can be used to determine if the mammal has ovarian tissue with an elevated level of ALDH. In some cases, a breast tissue biopsy obtained from a mammal (e.g., a human) can be used to determine if the mammal has breast tissue with an elevated level of ALDH activity, or an ovarian tissue biopsy obtained from a mammal (e.g., a human) can be used to determine if the mammal has ovarian tissue with an elevated level of ALDH activity. Tissue can be obtained from a mammal (e.g., a human) having chemotherapy-resistant cancer (e.g., paclitaxel-resistant breast cancer). In some cases, tissue can be obtained from a mammal (e.g., a human) having chemotherapy-resistant cancer (e.g., paclitaxel-resistant breast cancer) having previously received one or more chemotherapeutic agents (e.g., paclitaxel). In some cases, tissue can be obtained from a mammal (e.g., a human) having chemotherapy-resistant cancer (e.g., paclitaxel-resistant breast cancer) having previously received one or more chemotherapeutic agents (e.g., paclitaxel) and one or more NOTCH-3 targeted therapies (e.g., an anti-NOTCH3 antibody therapy, or a small hairpin RNA (shRNA) targeted to a NOTCH3 mRNA).

The term “elevated level” as used herein with respect to a level of NOTCH3 refers to a level of NOTCH3 present within a tissue (e.g., a breast or ovarian biopsy) that is greater (e.g., at least 10, 25, 35, 45, 50, 55, 65, 75, 80, 90, or 100 percent greater) than the median level of NOTCH3 present within a control tissue of comparable mammals. The term “elevated level” as used herein with respect to a level of ALDH refers to a level of ALDH present within a tissue (e.g., a breast or ovarian biopsy) that is greater (e.g., at least 10, 25, 35, 45, 50, 55, 65, 75, 80, 90, or 100 percent greater) than the median level of ALDH present within a control tissue of comparable mammals. The term “elevated level” as used herein with respect to a level of ALDH activity refers to a level of ALDH activity present within a tissue that is greater (e.g., at least 10, 25, 35, 45, 50, 55, 65, 75, 80, 90, or 100 percent greater) than the median level of NOTCH3 present within a control tissue of comparable mammals. Examples of such control tissue include, without limitation, tissue having a cancer that is not resistant to treatment with a chemotherapeutic agent, or tissue not having a cancer (e.g., healthy breast or ovarian tissue).

Once a mammal (e.g., a human) having chemotherapy-resistant cancer is identified as having an elevated level of NOTCH3 as described herein, the mammal can be classified as having chemotherapy-resistant cancer that includes the presence of an elevated level of NOTCH3. Once a mammal (e.g., a human) having chemotherapy-resistant cancer is identified as having an elevated level of ALDH as described herein, the mammal can be classified as having chemotherapy-resistant cancer that includes the presence of an elevated level of ALDH. Once a mammal (e.g., a human) having chemotherapy-resistant cancer is identified as having an elevated level of ALDH activity as described herein, the mammal can be classified as having chemotherapy-resistant cancer that includes the presence of an elevated level of ALDH. In some cases, a mammal (e.g., a human) having a chemotherapy-resistant cancer that is identified as having an elevated level of NOTCH3 as described herein can be classified as having chemotherapy-resistant cancer that includes chemotherapy-resistant tissue having an elevated level of a NOTCH3 polypeptide. In some cases, a mammal (e.g., a human) having a chemotherapy-resistant cancer that is identified as having an elevated level of ALDH as described herein can be classified as having chemotherapy-resistant cancer that includes chemotherapy-resistant tissue having an elevated level of an ALDH polypeptide. In some cases, a mammal (e.g., a human) having chemotherapy-resistant cancer that is identified as having an elevated level of ALDH activity as described herein can be classified as having chemotherapy-resistant cancer that includes chemotherapy-resistant tissue having an elevated level of ALDH activity.

As described herein, this document also provides methods and materials for treating a mammal having a cancer that is resistant to treatment with a chemotherapeutic agent. For example, a mammal (e.g., a human) having a chemotherapy-resistant cancer that is identified as having an elevated level of NOTCH3 as described herein can be treated with one or more NOTCH3 inhibitors. In another example, a mammal (e.g., a human) having a chemotherapy-resistant cancer that is identified as having an elevated level of ALDH as described herein can be treated with one or more NOTCH3 inhibitors. In another example, a mammal (e.g., a human) having a chemotherapy-resistant cancer that is identified as having an elevated level of ALDH activity as described herein can be treated with one or more NOTCH3 inhibitors. In yet another example, a mammal (e.g., a human) having a chemotherapy-resistant cancer that is identified as having an elevated level of NOTCH3 and as having an elevated level of ALDH activity as described herein can be treated with one or more NOTCH3 inhibitors. In some cases, a mammal (e.g., a human) having a chemotherapy-resistant cancer that is identified as having an elevated level of NOTCH3 as described herein can be administered, or instructed to self-administer, one or more NOTCH3 inhibitors to treat the chemotherapy-resistant cancer.

Any appropriate NOTCH3 inhibitor can be administered to a mammal (e.g., a mammal having chemotherapy-resistant cancer that was identified as having an elevated level of NOTCH3 and/or as having an elevated level of ALDH activity) to treat chemotherapy-resistant cancer. In some cases, a NOTCH-3 inhibitor used as described herein to treat cancer can reduce symptoms of the cancer within a mammal (e.g., cancer metastasis, pain, and/or overall mortality). Examples of NOTCH-3 inhibitors that can be used as described herein to treat chemotherapy-resistant cancer include, without limitation, antibodies that can bind specifically to NOTCH3 (e.g., without detectable binding to other proteins such as NOTCH1, NOTCH2, or NOTCH4), and shRNA molecules targeted to NOTCH3 mRNA. In some cases, for example, a humanized antibody such as AV-353 (AVEO Oncology; Boston, MA) can be administered to a mammal having a chemotherapy-resistant cancer (e.g., in an amount effective to reduce or inhibit NOTCH3 activity in the cancer cells). In some cases, a shRNA targeted to a NOTCH3 mRNA can be administered to a mammal having a chemotherapy-resistant cancer (e.g., in an amount effective to reduce NOTCH3 expression and/or to reduce or inhibit NOTCH3 activity in the cancer). Examples of shRNA sequences that can be used include, without limitation:

(SEQ ID NO: 1)
5′-AUCAAUGUUGACUUCACAGUU-3′,
(SEQ ID NO: 2)
5′-GCCAGAACUGUGAAGUCAATT-3′,
(SEQ ID NO: 3)
5′-GGACAUGCAGGAUAGCAAGGAGGG-3′,
(SEQ ID NO: 4)
5′-AGAUUAAUGAGGAUGACUGCGGCCC-3′,
and
(SEQ ID NO: 5)
5′-AGAUGGGACAUGUUCCAUAGCCUTG-3′.

In some cases, two or more (e.g., two, three, four, five, six, or more) NOTCH3 inhibitors can be administered to a mammal (e.g., (a) a mammal having chemotherapy-resistant cancer and identified as having an elevated level of NOTCH3); (b) a mammal having chemotherapy-resistant cancer and identified as having an elevated level of ALDH; (c) a mammal having chemotherapy-resistant cancer and identified as having an elevated level of ALDH activity; (d) a mammal having chemotherapy-resistant cancer and identified as having an elevated level of NOTCH3 and having an elevated level of ALDH activity; (e) a mammal having chemotherapy-resistant cancer and identified as having an elevated level of NOTCH3 and having an elevated level of ALDH, (f) a mammal having chemotherapy-resistant cancer and identified as having an elevated level of ALDH and having an elevated level of ALDH activity; or (g) a mammal having chemotherapy-resistant cancer and identified as having an elevated level of NOTCH3, having an elevated level of ALDH, and having an elevated level of ALDH activity) to treat the chemotherapy-resistant cancer. For example, two or more NOTCH3 inhibitors (e.g., an antibody such as AV-353 and a shRNA having a sequence set forth SEQ ID NO:1, SEQ ID NO:2, SEQ ID NO:3, SEQ ID NO:4, SEQ ID NO:5, or combinations thereof) can be administered to a mammal having chemotherapy-resistant cancer that was identified as having an elevated level of NOTCH3.

In some cases, a chemotherapeutic agent also can be administered to a mammal (e.g., a human) having a cancer that is resistant to treatment with a chemotherapeutic agent. In some cases, a chemotherapeutic agent can be administered with a NOTCH3 inhibitor (e.g., on the same day that a NOTCH3 inhibitor is administered). In some cases, a chemotherapeutic agent can be administered after a NOTCH3 inhibitor has been administered to the mammal. For example, a chemotherapeutic agent can be administered to a mammal (e.g., a human) from 1 day to 4 months after a NOTCH3 inhibitor was administered to the mammal. In some cases, a chemotherapeutic agent can be administered to a mammal (e.g., a human) from 1 to 3 days, 3 to 5 days, 5 to 7 days, 7 to 14 days, 14 to 28 days, 1 to 2 months, 2 to 3 months, 3 to 4 months, not more than 7 days, not more than 14 days, not more than 1 month, not more than 2 months, or not more than 3 months, or not more than 4 months after a NOTCH3 inhibitor was administered to the mammal. In some cases, a chemotherapeutic agent can be administered to a mammal (e.g., a human) from 1 hour to 4 months before a NOTCH3 inhibitor is administered to the mammal. For example, a chemotherapeutic agent can be administered to a mammal (e.g., a human) from 1 to 2 hours, 2 to 4 hours, 4 to 8 hours, 8 to 12 hours, 12 to 24 hours, 1 to 2 days, 2 to 3 days, 3 to 5 days, 5 to 7 days, not more than 24 hours, not more than 48 hours, not more than 72 hours, not more than 4 days, not more than 7 days, not more than 14 days, not more than 21 days, not more than 1 month, not more than 2 months, not more than 3 months, or not more than 4 months after a NOTCH3 inhibitor was administered to the mammal.

Any appropriate chemotherapeutic agent can be administered to a mammal (e.g., a mammal having a chemotherapy-resistant cancer that was identified as having an elevated level of NOTCH3 to treat the chemotherapy-resistant cancer. Any appropriate chemotherapeutic agent can be administered to a mammal (e.g., a mammal having chemotherapy-resistant cancer that was identified as having an elevated level of ALDH to treat the chemotherapy-resistant cancer. Any appropriate chemotherapeutic agent can be administered to a mammal (e.g., a mammal having chemotherapy-resistant cancer that was identified as having an elevated level of ALDH activity to treat the chemotherapy-resistant cancer. In some cases, a chemotherapeutic agent used as described herein to treat a chemotherapy-resistant cancer can reduce symptoms of cancer within a mammal (e.g., cancer metastasis, pain, and/or overall mortality). Examples of chemotherapeutic agents that can be used as described herein to treat cancer include, without limitation, paclitaxel, cabazitaxel, docetaxel, Nab-paclitaxel, vinblastine, vincristine, vinorelbine, altretamine, bendamustine, busulfan, carboplatin, carmustine, chlorambucil, cisplatin, cyclophosphamide, dacarbazine, ifosfamide, lomustine, mechlorethamine, melphalan, oxaliplatin, temozolomide, thiotepa, trabectedin, azacitidine, 5-fluorouracil, 6-mercaptopurine, capecitabine, cladribine, clofarabine, cytarabine, decitabine, floxuridine, fludarabine, gemcitabine, hydroxyurea, methotrexate, nelarabine, pemetrexed, pentostatin, pralatrexate, thioguanine, dunorubicin, doxorubicin, epirubicin, idarubicin, valrubicin, bleomycin, dactinomycin, mitomycin-C, mitoxantrone, irinotecan, topotecan, etoposide, and teniposide. In some cases, for example, paclitaxel, Nab-paclitaxel, or doxorubicin can be used as described herein to treat cancer.

In some cases, two or more (e.g., two, three, four, five, six, or more) chemotherapeutic agents can be administered to a mammal (e.g., (a) a mammal having chemotherapy-resistant cancer and identified as having an elevated level of NOTCH3; (b) a mammal having chemotherapy-resistant cancer and identified as having an elevated level of ALDH; (c) a mammal having chemotherapy-resistant cancer and identified as having an elevated level of ALDH activity; or (d) a mammal having chemotherapy-resistant cancer and identified as having an elevated level of NOTCH3 and having an elevated level of ALDH activity) to treat the chemotherapy-resistant cancer. For example, two chemotherapeutic agents can be administered to a mammal having chemotherapy-resistant cancer that was identified as having an elevated level of NOTCH3.

In some cases, one or more (e.g., two, three, four, five, six, or more) immune checkpoint inhibitors (e.g., inhibitors of PD-1, such as nivolumab, pembrolizumab, and cemiplimab; inhibitors of CTLA-4, such as ipilimumab and tremelimumab; and inhibitors of PD-L1, such as atezolizumab, avelumab, and durvalumab) can be administered to a mammal having a chemotherapy-resistant cancer (e.g., (a) a mammal having chemotherapy-resistant cancer and identified as having an elevated level of NOTCH3; (b) a mammal having chemotherapy-resistant cancer and identified as having an elevated level of ALDH; (c) a mammal having chemotherapy-resistant cancer and identified as having an elevated level of ALDH activity; or (d) a mammal having chemotherapy-resistant cancer and identified as having an elevated level of NOTCH3 and having an elevated level of ALDH activity) to treat the chemotherapy-resistant cancer. The one or more immune checkpoint inhibitors can be administered with a NOTCH3 inhibitor, with a chemotherapeutic agent, with both a NOTCH3 inhibitor and a chemotherapeutic agent, or separate from a NOTCH3 inhibitor and a chemotherapeutic agent. In some cases, an immune checkpoint inhibitor can be administered to a mammal (e.g., a human) along with a chemotherapeutic agent, after the mammal was administered a NOTCH3 inhibitor.

In some cases, one or more NOTCH3 inhibitors can be administered to a mammal once or multiple times over a period of time ranging from days to months. In some cases, one or more NOTCH3 inhibitors and one or more chemotherapeutic agents can be administered to a mammal once or multiple times over a period of time ranging from days to months to years. In some cases, one or more NOTCH3 inhibitors, or one or more NOTCH3 inhibitors and one or more chemotherapeutic agents, can be given to achieve remission of chemotherapy-resistant cancer, and then given during follow up periods to prevent relapse of the chemotherapy-resistant cancer.

In some cases, one or more NOTCH3 inhibitors and/or one or more chemotherapeutic agents can be formulated into a pharmaceutically acceptable composition for administration to a mammal (e.g., a human) having a chemotherapy-resistant cancer, to reduce symptoms of the cancer within that mammal (e.g., tumor metastasis, pain, and/or overall mortality). For example, a therapeutically effective amount of one or more NOTCH3 inhibitors and/or one or more chemotherapeutic agents can be formulated together with one or more pharmaceutically acceptable carriers (additives) and/or diluents. In some cases, a therapeutically effective amount of one or more NOTCH3 inhibitors and/or one or more chemotherapeutic agents can be individually formulated with one or more pharmaceutically acceptable carriers (additives) and/or diluents. A pharmaceutical composition can be formulated for administration in solid or liquid form including, without limitation, in the form of sterile solutions, suspensions, sustained-release formulations, tablets, capsules, pills, powders, or granules.

Pharmaceutically acceptable carriers, fillers, and vehicles that may be used in a pharmaceutical composition described herein include, without limitation, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

A pharmaceutical composition containing one or more NOTCH3 inhibitors and/or one or more chemotherapeutic agents can be designed for oral or parenteral (including subcutaneous, intramuscular, intravenous, and intradermal) administration. When being administered orally, a pharmaceutical composition can be in the form of a pill, tablet, or capsule. Compositions suitable for parenteral administration include aqueous and non-aqueous sterile injection solutions that can contain anti-oxidants, buffers, bacteriostats, and solutes that render the formulation isotonic with the blood of the intended recipient.

The formulations can be presented in unit-dose or multi-dose containers, for example, sealed ampules and vials, and may be stored in a freeze dried (lyophilized) condition requiring only the addition of the sterile liquid carrier, for example, water for injections, immediately prior to use. Extemporaneous injection solutions and suspensions may be prepared from sterile powders, granules, and tablets.

In some cases, a pharmaceutically acceptable composition including one or more NOTCH3 inhibitors and/or one or more chemotherapeutic agents can be administered locally or systemically. For example, a composition provided herein can be administered locally by intravenous injection or blood infusion. In some cases, a composition provided herein can be administered systemically, orally, or by injection to a mammal (e.g., a human).

Effective doses can vary depending on the severity of the chemotherapy-resistant cancer, the route of administration, the age and general health condition of the subject, excipient usage, the possibility of co-usage with other therapeutic treatments, and the judgment of the treating clinician. An effective amount of a composition containing one or more NOTCH3 inhibitors described herein can be any amount that results in a reduced level of NOTCH3 expression and/or a reduced level of NOTCH3 activity in cancer cells within a mammal (e.g., a human), without producing severe toxicity to the mammal. The term “reduced level” as used herein with respect to a level of NOTCH3 expression refers to a level of NOTCH3 present within a tissue (e.g., a breast or ovarian biopsy) that is less than (e.g., at least 10, 25, 35, 45, 50, 55, 65, 75, 80, 90, or 100 percent less) than the level of NOTCH3 present within a comparable sample of the tissue that was obtained prior to treatment with a NOTCH3 inhibitor.

Any appropriate method can be used to measure a level of NOTCH3 in cancer cells after administration of a NOTCH3 inhibitor, to determine whether the amount of NOTCH3 expression is reduced as compared to the level of expression prior to the administration. For example, IHC techniques, IF techniques, mass spectrometry-based proteomics, or Western blot techniques can be used to determine a level of NOTCH3 expression in a tissue sample containing cancer cells. In some cases, a tissue sample (e.g., a breast biopsy or an ovarian biopsy) obtained from a mammal can be stained using an anti-NOTCH3 antibody to determine a level of NOTCH3 polypeptide in the tissue sample. In some cases, mRNA levels can be used as an indicator of polypeptide levels, and can be used to determine a level of NOTCH3 in a tissue (e.g., breast tissue or ovarian tissue). Any appropriate method of quantifying mRNA can be used. Examples of methods of quantifying mRNA include, without limitation, qRT-PCR, RNA-sequencing, microfluidic capillary electrophoresis, and in situ hybridization.

In some cases, an effective amount of a composition containing one or more NOTCH3 inhibitors and/or one or more chemotherapeutic agents described herein can be any amount that reduces the number of cancer cells (e.g., by at least 5, 10, 25, 35, 45, 50, 55, 65, 75, 80, 90, or 100 percent) within a mammal (e.g., a human), without producing severe toxicity in the mammal. In some cases, an effective amount of a composition containing one or more NOTCH3 inhibitors and/or one or more chemotherapeutic agents described herein can be any amount that reduces the size (e.g., by at least 5, 10, 25, 35, 45, 50, 55, 65, 75, 80, 90, or 100 percent) of a tumor containing chemotherapy-resistant cancer cells within a mammal (e.g., a human), without producing severe toxicity to the mammal.

For example, an effective amount of an anti-NOTCH3 antibody (e.g., AV-353) can be from about 1 mg/Kg to about 400 mg/Kg (e.g., about 1 to about 10 mg/Kg, about 10 to about 20 mg/Kg, about 20 to 30 mg/Kg, about 30 to about 40 mg/Kg, about 10 to about 40 mg/Kg, about 40 to about 50 mg/Kg, about 50 to about 100 mg/Kg, about 100 to about 200 mg/Kg, about 200 to about 300 mg/Kg, or about 300 to about 400 mg/Kg). For example, an effective amount of a shRNA targeted to a NOTCH3 mRNA (e.g., a shRNA having a sequence set forth in SEQ ID NO:1, SEQ ID NO:2, SEQ ID NO:3, SEQ ID NO:4, or SEQ ID NO:5) can be from about 0.25 mg/Kg to about 50 mg/Kg (e.g., about 0.25 to about 0.5 mg/Kg, about 0.5 to about 1 mg/Kg, about 1 to about 2 mg/Kg, about 2 to about 3 mg/Kg, about 2.5 to about 5 mg/Kg, about 3 to about 4 mg/Kg, about 4 to about 5 mg/Kg, about 5 to about 10 mg/Kg, about 10 to about 20 mg/Kg, about 20 to about 30 mg/Kg, about 30 to about 40 mg/Kg, or about 40 to about 50 mg/Kg). For example, an effective amount of a chemotherapeutic agent (e.g., paclitaxel, Nab-paclitaxel, or doxorubicin) can be from about 10 mg/Kg to about 20 mg/Kg (e.g., about 1 to about 5 mg/Kg, about 5 to about 10 mg/Kg, about 10 to about 15 mg/Kg, about 10 to about 20 mg/Kg, about 15 to about 20 mg/Kg, about 20 to about 30 mg/Kg, about 30 to about 50 mg/Kg, about 50 to about 100 mg/Kg, about 100 to about 150 mg/Kg, or about 150 to about 200 mg/Kg). In some cases, an effective amount of a chemotherapeutic agent (e.g., paclitaxel, Nab-paclitaxel, or doxorubicin) can be from about 30 mg/m² to about 90 mg/m² (e.g., about 30 to about 40 mg/m², about 40 to about 50 mg/m², about 50 to about 60 mg/m², about 60 to about 70 mg/m², about 70 to about 80 mg/m², or about 80 to about 90 mg/m²).

If a particular mammal fails to respond to a particular amount, then the amount of the NOTCH3 inhibitor can be increased by, for example, two fold. If a particular mammal fails to respond to a particular amount, then the amount of the chemotherapeutic agent can be increased by, for example, two fold. After receiving the higher amount of either one or both of the one or more NOTCH3 inhibitor and/or one or more chemotherapeutic agents, the mammal can be monitored for both responsiveness to the treatment and toxicity symptoms, and adjustments made accordingly. The effective amount can remain constant or can be adjusted as a sliding scale or variable dose depending on the mammal's response to treatment. Various factors can influence the actual effective amount used for a particular application. For example, the frequency of administration, duration of treatment, use of multiple treatment agents, route of administration, and severity of the condition (e.g., chemotherapy-resistant cancer) may require an increase or decrease in the actual effective amount administered.

The frequency of administration of one/or more NOTCH3 inhibitors and/or one or more chemotherapeutic agents described herein can be any amount that reduces the number of chemotherapy-resistant cancer cells and/or reduces the size of a tumor containing chemotherapy-resistant cancer cells within a mammal (e.g., a human) without producing significant toxicity to the mammal. For example, the frequency of administration of a NOTCH3 inhibitor (e.g., AV-353) can be from about once a day to about once a week (e.g., once every other day) or from about once a day to about once a month. For example, the frequency of administration of a chemotherapeutic agent can be from about once a day to about once a week about once a month (e.g., from about once a week to about once every other week). The frequency of administration of one/or more NOTCH3 inhibitors and one or more chemotherapeutic agents described herein can remain constant or can be variable during the duration of treatment. A course of treatment with a composition containing one/or more NOTCH3 inhibitors and/or one or more chemotherapeutic agents described herein can include rest periods. For example, a composition containing one/or more NOTCH3 inhibitors and/or one or more chemotherapeutic agents described herein can be administered daily over a one-week period followed by a one-week rest period, and such a regimen can be repeated multiple times. As with the effective amount, various factors can influence the actual frequency of administration used for a particular application. For example, the effective amount, duration of treatment, use of multiple treatment agents, route of administration, and severity of the condition (e.g., cancer) may require an increase or decrease in administration frequency.

An effective duration for administering a composition containing one/or more NOTCH3 inhibitors and/or one or more chemotherapeutic agents described herein can be any duration that reduces the number of chemotherapy-resistant cancer cells and/or reduces the size of a tumor containing chemotherapy-resistant cancer cells within a mammal (e.g., a human) without producing significant toxicity to the mammal. In some cases, the effective duration can vary from several days to several months. Multiple factors can influence the actual effective duration used for a particular treatment. For example, an effective duration can vary with the frequency of administration, effective amount, use of multiple treatment agents, route of administration, and severity of the condition being treated.

In some cases, a course of treatment and/or the severity of one or more symptoms related to the condition being treated (e.g., chemotherapy-resistant cancer) can be monitored. Any appropriate method can be used to determine whether or not a mammal having chemotherapy-resistant cancer is being treated. For example, clinical scanning techniques (e.g., computed tomography (CT), positron emission tomography (PET)/CT, bone scan, and magnetic resonance imaging (MRI)) can be used to determine the presence or absence of chemotherapy-resistant cancer within a mammal (e.g., a human) being treated. In some cases, a mammal can be monitored by determining the level of NOTCH3, the level of ALDH activity, or the level of PD-L1 in a tissue sample (e.g., a tissue sample obtained from a location associated with cancer in the mammal) to determine whether the level of NOTCH3 expression, the level of ALDH activity, or the level of PD-L1 expression is reduced as compared to a level determined prior to treatment, or as compared to a level determined at an earlier time point after treatment. A reduced level of NOTCH3 expression, a reduced level of ALDH activity, and/or a reduced level of PD-L1 expression can indicate effective treatment.

### EXEMPLARY EMBODIMENTS

Embodiment 1 is a method for treating a mammal having a cancer identified as being resistant to a chemotherapeutic agent, wherein the method comprises administering a NOTCH3 inhibitor to the mammal, thereby increasing the susceptibility of the cancer to the chemotherapeutic agent, and administering the chemotherapeutic agent to the mammal.

Embodiment 2 is the method of embodiment 1, wherein the cancer is a metastatic cancer.

Embodiment 3 is the method of embodiment 1 or embodiment 2, wherein the cancer comprises NOTCH3⁺ cells.

Embodiment 4 is the method of embodiment 3, wherein the method further comprises, prior to administering the NOTCH3 inhibitor, detecting the presence of the NOTCH3⁺ cells.

Embodiment 5 is the method of any one of embodiments 1 to 4, wherein the cancer comprises NOTCH3 over-expressing cells.

Embodiment 6 is the method of embodiment 5, wherein the method further comprises, prior to administering the NOTCH3 inhibitor, detecting the presence of the NOTCH3 over-expressing cells.

Embodiment 7 is the method of any one of embodiments 1 to 6, wherein the cancer comprises ALDH⁺ cells.

Embodiment 8 is the method of embodiment 7, wherein the method further comprises, prior to administering the NOTCH3 inhibitor, detecting the presence of the ALDH⁺ cells.

Embodiment 9 is the method of any one of embodiments 1 to 8, wherein the cancer comprises ALDH over-expressing cells.

Embodiment 10 is the method of embodiment 9, wherein the method further comprises, prior to administering the NOTCH3 inhibitor, detecting the presence of the ALDH over-expressing cells.

Embodiment 11 is the method of any one of embodiments 1 to 10, wherein the cancer comprises Epithelial to Mesenchymal Transition⁺ (EMT⁺) cells.

Embodiment 12 is the method of embodiment 11, wherein the method further comprises, prior to administering the NOTCH3 inhibitor, detecting the presence of the EMT⁺ cells.

Embodiment 13 is the method of any one of embodiments 1 to 12, wherein the cancer comprises cancer stem-like cells.

Embodiment 14 is the method of embodiment 13, wherein the method further comprises, prior to administering the NOTCH3 inhibitor, detecting the presence of the cancer stem-like cells.

Embodiment 15 is the method of any one of embodiments 1 to 14, wherein the cancer is triple negative breast cancer (TNBC), ovarian cancer, or another solid tumor with elevated NOTCH3 expression.

Embodiment 16 is the method of any one of embodiments 1 to 15, wherein the cancer is NOTCH3⁺ TNBC, NOTCH3⁺ ovarian cancer, ALDH⁺ TNBC, ALDH⁺ ovarian cancer, ALDH⁺ and NOTCH3⁺ TNBC, or ALDH⁺ and NOTCH3⁺ ovarian cancer.

Embodiment 17 is the method of any one of embodiments 1 to 16, wherein the NOTCH3 inhibitor comprises a shRNA targeted to NOTCH3.

Embodiment 18 is the method of embodiment 17, wherein the shRNA comprises the nucleotide sequence set forth in any of SEQ ID NOS:1 to 5.

Embodiment 19 is the method of any one of embodiments 1 to 16, wherein the NOTCH3 inhibitor comprises an antibody.

Embodiment 20 is the method of embodiment 19, wherein the antibody is AV-353.

Embodiment 21 is the method of any one of embodiments 1 to 20, wherein the mammal was treated with the chemotherapeutic agent without a complete response more than 4 months prior to administering the NOTCH3 inhibitor.

Embodiment 22 is the method of any one of embodiments 1 to 21, comprising administering the NOTCH3 inhibitor no more than 4 months prior to administering the chemotherapeutic agent.

Embodiment 23 is the method of any one of embodiments 1 to 21, comprising administering the chemotherapeutic agent no more than 4 months prior to administering the NOTCH3 inhibitor.

Embodiment 24 is the method of any one of embodiments 1 to 21, comprising administering the NOTCH3 inhibitor with the chemotherapeutic agent.

Embodiment 25 is the method of any one of embodiments 1 to 24, wherein the chemotherapeutic agent is selected from the group consisting of paclitaxel, cabazitaxel, docetaxel, Nab-paclitaxel, vinblastine, vincristine, vinorelbine, altretamine, bendamustine, busulfan, carboplatin, carmustine, chlorambucil, cisplatin, cyclophosphamide, dacarbazine, ifosfamide, lomustine, mechlorethamine, melphalan, oxaliplatin, temozolomide, thiotepa, trabectedin, azacitidine, 5-fluorouracil, 6-mercaptopurine, capecitabine, cladribine, clofarabine, cytarabine, decitabine, floxuridine, fludarabine, gemcitabine, hydroxyurea, methotrexate, nelarabine, pemetrexed, pentostatin, pralatrexate, thioguanine, dunorubicin, doxorubicin, epirubicin, idarubicin, valrubicin, bleomycin, dactinomycin, mitomycin-C, mitoxantrone, irinotecan, topotecan, etoposide, and teniposide.

Embodiment 26 is the method of any one of embodiments 1 to 25, further comprising, after administering the NOTCH3 inhibitor, monitoring NOTCH3 levels in the mammal, wherein a decrease in the NOTCH3 levels indicates successful treatment with the NOTCH3 inhibitor.

Embodiment 27 is the method of any one of embodiments 1 to 26, further comprising, after administering the NOTCH3 inhibitor, monitoring PD-L1 levels in the mammal, wherein a decrease in the PD-L1 levels indicates successful treatment with the NOTCH3 inhibitor.

Embodiment 28 is the method of any one of embodiments 1 to 27, further comprising administering to the mammal a checkpoint inhibitor.

Embodiment 29 is the method of embodiment 28, wherein the checkpoint inhibitor comprises nivolumab, pembrolizumab, cemiplimab, ipilimumab, tremelimumab, atezolizumab, avelumab, or durvalumab.

Embodiment 30 is the method of embodiment 28, wherein the checkpoint inhibitor comprises pembrolizumab.

Embodiment 31 is a method for identifying a mammal as having a cancer resistant to a chemotherapeutic agent and as being likely to respond to treatment with a NOTCH3 inhibitor and the chemotherapeutic agent, wherein the method comprises measuring a level of NOTCH3 in cells from the cancer, and when the measured level of NOTCH3 is elevated as compared to a control level of NOTCH3 in normal tissue, identifying the mammal as being likely to respond to treatment with the NOTCH3 inhibitor and the chemotherapeutic agent as opposed to treatment with the chemotherapeutic agent in the absence of the NOTCH3 inhibitor.

Embodiment 32 is the method of embodiment 31, wherein the cancer is a metastatic cancer.

Embodiment 33 is the method of embodiment 31 or embodiment 32, wherein the cancer is TNBC, ovarian cancer, or another solid tumor with elevated NOTCH expression.

Embodiment 34 is the method of any one of embodiments 31 to 33, wherein the mammal was treated with the chemotherapeutic agent without a complete response more than 4 months prior to administering the NOTCH3 inhibitor.

Embodiment 35 is the method of any one of embodiments 31 to 34, further comprising administering the NOTCH3 inhibitor and the chemotherapeutic agent to the mammal.

Embodiment 36 is the method of embodiment 35, comprising administering the NOTCH3 inhibitor no more than 4 months prior to administering the chemotherapeutic agent.

Embodiment 37 is the method of embodiment 35, comprising administering the chemotherapeutic agent no more than 4 months prior to administering the NOTCH3 inhibitor.

Embodiment 38 is the method of embodiment 35, comprising administering the NOTCH3 inhibitor with the chemotherapeutic agent.

Embodiment 39 is the method of any one of embodiments 35 to 38, wherein the chemotherapeutic agent is selected from the group consisting of paclitaxel, cabazitaxel, docetaxel, Nab-paclitaxel, vinblastine, vincristine, vinorelbine, altretamine, bendamustine, busulfan, carboplatin, carmustine, chlorambucil, cisplatin, cyclophosphamide, dacarbazine, ifosfamide, lomustine, mechlorethamine, melphalan, oxaliplatin, temozolomide, thiotepa, trabectedin, azacitidine, 5-fluorouracil, 6-mercaptopurine, capecitabine, cladribine, clofarabine, cytarabine, decitabine, floxuridine, fludarabine, gemcitabine, hydroxyurea, methotrexate, nelarabine, pemetrexed, pentostatin, pralatrexate, thioguanine, dunorubicin, doxorubicin, epirubicin, idarubicin, valrubicin, bleomycin, dactinomycin, mitomycin-C, mitoxantrone, irinotecan, topotecan, etoposide, and teniposide.

Embodiment 40 is the method of any one of embodiments 35 to 39, further comprising, after administering the NOTCH3 inhibitor, monitoring NOTCH3 levels in the mammal, wherein a decrease in the NOTCH3 levels indicates successful treatment with the NOTCH3 inhibitor.

Embodiment 41 is a method for identifying a mammal as having a cancer resistant to a chemotherapeutic agent and as being likely to respond to treatment with a NOTCH3 inhibitor and the chemotherapeutic agent, wherein the method comprises measuring a level of ALDH activity in cells from the cancer, and when the measured level of ALDH activity is elevated as compared to a control level of ALDH, identifying the mammal as being likely to respond to treatment with the NOTCH3 inhibitor and the chemotherapeutic agent as opposed to treatment with the chemotherapeutic agent in the absence of the NOTCH3 inhibitor.

Embodiment 42 is the method of embodiment 41, wherein the cancer is a metastatic cancer.

Embodiment 43 is the method of embodiment 41 or embodiment 42, wherein the cancer is TNBC, ovarian cancer, or another solid tumor with elevated NOTCH3 expression.

Embodiment 44 is the method of any one of embodiments 41 to 43, wherein the mammal was treated with the chemotherapeutic agent without a complete response more than 4 months prior to administering the NOTCH3 inhibitor.

Embodiment 45 is the method of any one of embodiments 41 to 44, further comprising administering the NOTCH3 inhibitor and the chemotherapeutic agent to the mammal.

Embodiment 46 is the method of embodiment 45, comprising administering the NOTCH3 inhibitor no more than 4 months prior to administering the chemotherapeutic agent.

Embodiment 47 is the method of embodiment 45, comprising administering the chemotherapeutic agent no more than 4 months prior to administering the NOTCH3 inhibitor.

Embodiment 48 is the method of embodiment 45, comprising administering the NOTCH3 inhibitor with the chemotherapeutic agent.

Embodiment 49 is the method of any one of embodiments 45 to 48, wherein the chemotherapeutic agent is selected from the group consisting of paclitaxel, cabazitaxel, docetaxel, Nab-paclitaxel, vinblastine, vincristine, vinorelbine, altretamine, bendamustine, busulfan, carboplatin, carmustine, chlorambucil, cisplatin, cyclophosphamide, dacarbazine, ifosfamide, lomustine, mechlorethamine, melphalan, oxaliplatin, temozolomide, thiotepa, trabectedin, azacitidine, 5-fluorouracil, 6-mercaptopurine, capecitabine, cladribine, clofarabine, cytarabine, decitabine, floxuridine, fludarabine, gemcitabine, hydroxyurea, methotrexate, nelarabine, pemetrexed, pentostatin, pralatrexate, thioguanine, dunorubicin, doxorubicin, epirubicin, idarubicin, valrubicin, bleomycin, dactinomycin, mitomycin-C, mitoxantrone, irinotecan, topotecan, etoposide, and teniposide.

Embodiment 50 is the method of any one of embodiments 45 to 49, further comprising, after administering the NOTCH3 inhibitor, monitoring NOTCH3 levels in the mammal, wherein a decrease in the NOTCH3 levels indicates successful treatment with the NOTCH3 inhibitor.

Embodiment 51 is a method for monitoring treatment of a mammal having a cancer resistant to a chemotherapeutic agent, wherein the method comprises identifying the mammal as having an elevated level of NOTCH3 in cells from the cancer, administering a NOTCH3 inhibitor to the mammal, and measuring a post-treatment level of NOTCH3 in cells of the cancer after the administering, wherein a decrease in the post-treatment level of NOTCH3 as compared to the elevated level of NOTCH3 indicates successful treatment of the mammal.

Embodiment 52 is the method of embodiment 51, wherein the cancer is a metastatic cancer.

Embodiment 53 is the method of embodiment 51 or embodiment 52, wherein the cancer is TNBC, ovarian cancer, or another solid tumor with elevated NOTCH3 expression.

Embodiment 54 is the method of any one of embodiments 51 to 53, wherein the cancer is NOTCH3⁺ TNBC, NOTCH3⁺ ovarian cancer, ALDH⁺ TNBC, ALDH⁺ ovarian cancer, ALDH⁺ and NOTCH3⁺ TNBC, or ALDH⁺ and NOTCH3⁺ ovarian cancer.

Embodiment 55 is the method of any one of embodiments 51 to 54, wherein the NOTCH3 inhibitor comprises a shRNA targeted to NOTCH3.

Embodiment 56 is the method of embodiment 55, wherein the shRNA comprises the nucleotide sequence set forth in any of SEQ ID NOS:1 to 5.

Embodiment 57 is the method of any one of embodiments 51 to 54, wherein the NOTCH3 inhibitor comprises an antibody.

Embodiment 58 is the method of embodiment 57, wherein the antibody is AV-353.

Embodiment 59 is the method of any one of embodiments 51 to 58, wherein the mammal was treated with the chemotherapeutic agent without a complete response more than 4 months prior to administering the NOTCH3 inhibitor.

Embodiment 60 is the method of any one of embodiments 51 to 59, further comprising administering the chemotherapeutic agent to the mammal.

Embodiment 61 is the method of embodiment 60, comprising administering the NOTCH3 inhibitor no more than 4 months prior to administering the chemotherapeutic agent.

Embodiment 62 is the method of embodiment 60, comprising administering the chemotherapeutic agent no more than 4 months prior to administering the NOTCH3 inhibitor.

Embodiment 63 is the method of embodiment 60, comprising administering the NOTCH3 inhibitor with the chemotherapeutic agent.

Embodiment 64 is the method of any one of embodiments 60 to 63, wherein the chemotherapeutic agent is selected from the group consisting of paclitaxel, cabazitaxel, docetaxel, Nab-paclitaxel, vinblastine, vincristine, vinorelbine, altretamine, bendamustine, busulfan, carboplatin, carmustine, chlorambucil, cisplatin, cyclophosphamide, dacarbazine, ifosfamide, lomustine, mechlorethamine, melphalan, oxaliplatin, temozolomide, thiotepa, trabectedin, azacitidine, 5-fluorouracil, 6-mercaptopurine, capecitabine, cladribine, clofarabine, cytarabine, decitabine, floxuridine, fludarabine, gemcitabine, hydroxyurea, methotrexate, nelarabine, pemetrexed, pentostatin, pralatrexate, thioguanine, dunorubicin, doxorubicin, epirubicin, idarubicin, valrubicin, bleomycin, dactinomycin, mitomycin-C, mitoxantrone, irinotecan, topotecan, etoposide, and teniposide.

Embodiment 65 is a method for treating a mammal having a cancer identified as being resistant to a chemotherapeutic agent, wherein the method comprises administering a NOTCH3 inhibitor to the mammal, thereby increasing the susceptibility of the cancer to the chemotherapeutic agent.

Embodiment 66 is the method of embodiment 65, wherein the cancer is a metastatic cancer.

Embodiment 67 is the method of embodiment 65 or embodiment 66, wherein the cancer comprises NOTCH3⁺ cells.

Embodiment 68 is the method of embodiment 67, wherein the method further comprises, prior to administering the NOTCH3 inhibitor, detecting the presence of the NOTCH3⁺ cells.

Embodiment 69 is the method of any one of embodiments 65 to 68, wherein the cancer comprises NOTCH3 over-expressing cells.

Embodiment 70 is the method of embodiment 69, wherein the method further comprises, prior to administering the NOTCH3 inhibitor, detecting the presence of the NOTCH3 over-expressing cells.

Embodiment 71 is the method of any one of embodiments 65 to 70, wherein the cancer comprises ALDH⁺ cells.

Embodiment 72 is the method of embodiment 71, wherein the method further comprises, prior to administering the NOTCH3 inhibitor, detecting the presence of the ALDH⁺ cells.

Embodiment 73 is the method of any one of embodiments 65 to 72, wherein the cancer comprises ALDH over-expressing cells.

Embodiment 74 is the method of embodiment 73, wherein the method further comprises, prior to administering the NOTCH3 inhibitor, detecting the presence of the ALDH over-expressing cells.

Embodiment 75 is the method of any one of embodiments 65 to 74, wherein the cancer comprises EMT⁺ cells.

Embodiment 76 is the method of embodiment 75, wherein the method further comprises, prior to administering the NOTCH3 inhibitor, detecting the presence of the EMT⁺ cells.

Embodiment 77 is the method of any one of embodiments 65 to 76, wherein the cancer comprises cancer stem-like cells.

Embodiment 78 is the method of embodiment 77, wherein the method further comprises, prior to administering the NOTCH3 inhibitor, detecting the presence of the cancer stem-like cells.

Embodiment 79 is the method of any one of embodiments 65 to 78, wherein the cancer is TNBC, ovarian cancer, or another solid tumor with elevated NOTCH3 expression.

Embodiment 80 is the method of any one of embodiments 65 to 79, wherein the cancer is NOTCH3⁺ TNBC, NOTCH3⁺ ovarian cancer, ALDH⁺ TNBC, ALDH⁺ ovarian cancer, ALDH⁺ and NOTCH3⁺ TNBC, or ALDH⁺ and NOTCH3⁺ ovarian cancer.

Embodiment 81 is the method of any one of embodiments 65 to 80, wherein the NOTCH3 inhibitor comprises a shRNA targeted to NOTCH3.

Embodiment 82 is the method of embodiment 81, wherein the shRNA comprises the nucleotide sequence set forth in any of SEQ ID NOs:1 to 5.

Embodiment 83 is the method of any one of embodiments 65 to 82, wherein the NOTCH3 inhibitor comprises an antibody.

Embodiment 84 is the method of embodiment 83, wherein the antibody is AV-353.

Embodiment 85 is the method of any one of embodiments 65 to 84, wherein the mammal was treated with the chemotherapeutic agent without a complete response more than 4 months prior to administering the NOTCH3 inhibitor.

Embodiment 86 is the method of any one of embodiments 65 to 85, further comprising, after administering the NOTCH3 inhibitor, monitoring NOTCH3 levels in the mammal, wherein a decrease in the NOTCH3 levels indicates successful treatment with the NOTCH3 inhibitor.

Embodiment 87 is the method of any one of embodiments 65 to 86, further comprising, after administering the NOTCH3 inhibitor, monitoring PD-L1 levels in the mammal, wherein a decrease in the PD-L1 levels indicates successful treatment with the NOTCH3 inhibitor.

Embodiment 88 is the method of any one of embodiments 65 to 87, further comprising administering to the mammal a checkpoint inhibitor.

Embodiment 89 is the method of embodiment 88, wherein the checkpoint inhibitor comprises nivolumab, pembrolizumab, cemiplimab, ipilimumab, tremelimumab, atezolizumab, avelumab, or durvalumab.

Embodiment 90 is the method of embodiment 88, wherein the checkpoint inhibitor comprises pembrolizumab.

The invention will be further described in the following examples, which do not limit the scope of the invention described in the claims.

### EXAMPLES

### Example 1—Selective Pharmacologic Targeting of Notch3 Inhibits Stemness and Immune Evasion Capacity in Triple Negative Breast Cancer

### Materials and Methods

Human Breast Cancer Cell Lines: The human breast cancer cell lines MDA-MB 231 were obtained from ATCC (Manassas, VA). SUM149-PT breast cancer cells were obtained from the Couch laboratory (Mayo Clinic). All cell lines were maintained in DMEM medium containing 5 mM glutamine, 1% penicillin/streptomycin and 10% FBS at 37° C. in 5% CO₂ atmosphere. PDX-derived TNBC cells (M14, M25 and M40) were established from metastatic PDX models in the Sarkaria laboratory (Mayo Clinic Cancer Center).

Mammospheres Formation: Human breast cancer cells were plated in ultra-low attachment 24-well and 96-well culture dishes in 100 μL of MAMMOCULT™ medium (STEMCELL™ Technologies; Vancouver, BC, Canada). Medium was added every 2 days for a maximum of 8 days. Mammospheres were trypsinized and expanded every 8 days to form tertiary mammospheres, and their growth was recorded through a digital camera (Nikon).

Real-Time Cell Proliferation, Cytotoxicity and Apoptosis Assays: 5,000 cells were plated in Costar 94-well plates, treated with Docetaxel (DTX) and/or AV-353 (as monotherapy or in combination). Cell proliferation was monitored in real-time by GFP or RFP fluorescence intensity, and Red Annexin-V was used as an early marker of apoptosis. Apoptotic cells were quantified in real-time using INCUCYTE® S3 (BioEssen). Experiments were performed in triplicate (±S.D.).

Immunoblot, Immunofluorescence and FACS Assays: Immunofluorescence and FACS assays were performed as described elsewhere (D'Assoro et al., Oncogene 2014, 33(5):599-610).

Lenti-vectors Targeting NOTCH3: Scrambled (control) and GFP-tagged NOTCH3 shRNA lenti-vectors were obtained from OriGene Technologies (Rockville, MD) and used according to the manufacturer's instructions.

ALDH Activity Assay: ALDH activity was detected by FACS analysis using the ALDEFLUOR™ assay kit (STEMCELL™ Technologies) as described elsewhere (Jalalirad et al., Oncogene. 2021, 40(14):2509-2523). Results were derived from three independent experiments with comparable outcomes (±S.D.).

Breast lumor Xenografts: Breast tumor xenografts were established as described elsewhere (Jalalirad et al., supra).

Scientific Rigor and Statistical Analysis: Experiments were run in triplicate or for three independent runs (±S.D.). The average read-out of the triplicates from each run was determined, and a 95% t-confidence interval for the difference between was constructed. Different breast cancer cell lines were employed in these studies to increase the power to detect the effect size. The nonparametric Mann-Whitney t test (Statview software) was used to determine the significance of the relative tumor volumes for treated versus untreated tumor xenograft groups. Using an initial sample size of 5 animals per group with an eventual increase to a sample size of 10 animals per group if experimental outcomes warranted, a two-sided (alpha=0.05), two sample t-test for assessing whether the difference in mean tumor burden differed significantly between a particular pair of treatment groups had a power of 90% to detect a difference of 1.6 standard deviation (SD). For each xenograft (treated and control groups), the difference in the percentage of organ metastatic burden was assessed. Animals were examined every day and body weight and primary tumor size was measured 3 times per week.

### Results

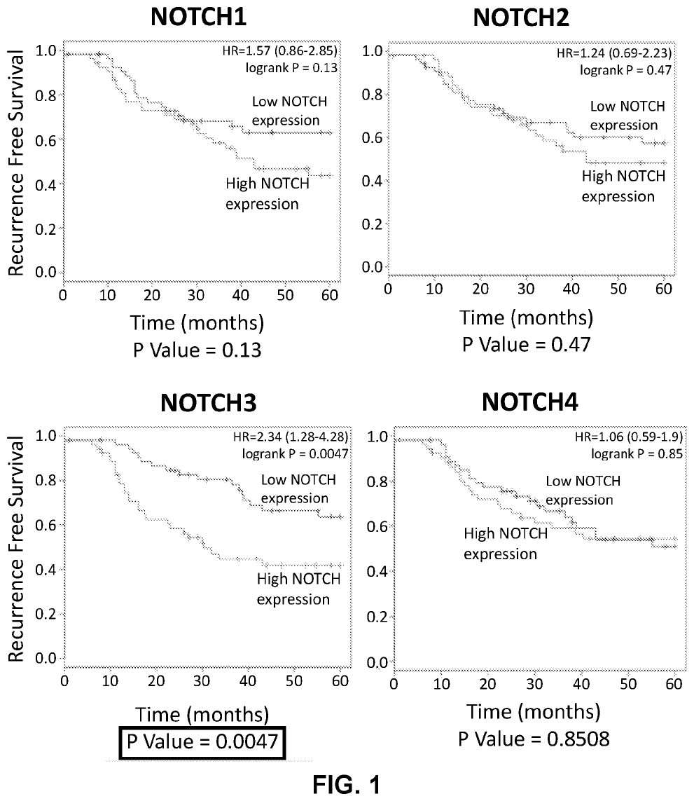

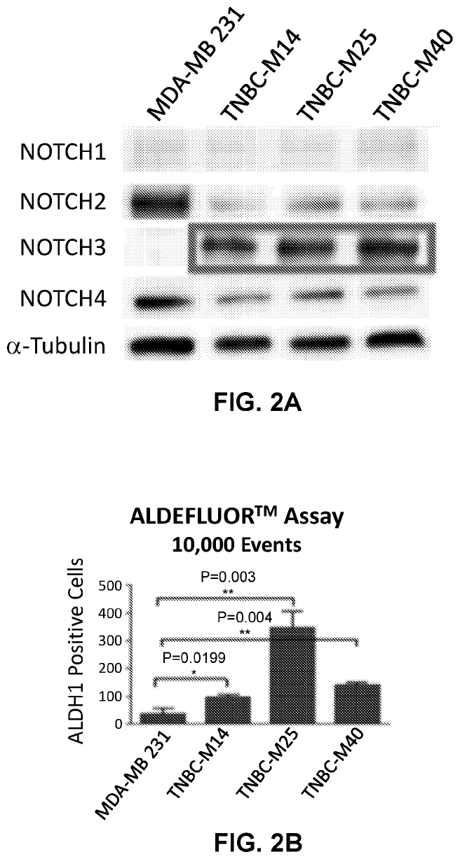

NOTCH3 mRNA expression is an indicator of poor prognosis in TNBC patients: To define the linkage between NOTCH mRNA expression and shorter recurrence-free survival (RFS), a selected cohort of 107 patients with lymph node⁺ TNBC from a publicly available clinical microarray database (kmplot.com) was analyzed. Only high NOTCH3 mRNA expression was significantly linked to reduced RFS (FIG. 1), indicating that NOTCH3 expression may be involved in TNBC progression. 3D-Mammospheres established from patient derived xenografts (PDXs) express high levels of NOTCH3 and have high ALDH activity: Ex vivo 3D-Mammospheres (MPS) were established using TNBC cells (TNBC-M14, TNBC-M25, and TNBC-M40) isolated from metastatic PDXs. TNBC-M14, TNBC-M25, and TNBC-M40 3D-MPS all showed NOTCH3 overexpression, as compared to MDA-MB 231 3D-MPS used as a control (FIG. 2A). TNBC-M14, TNBC-M25, and TNBC-M40 3D-MPS also showed higher ALDH activity than MDA-MB 231 (FIG. 2B).

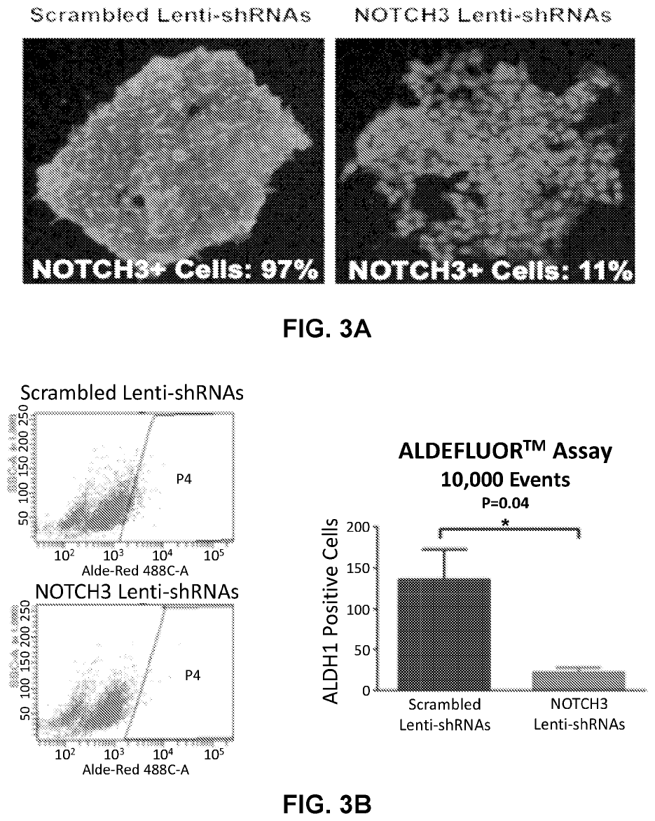

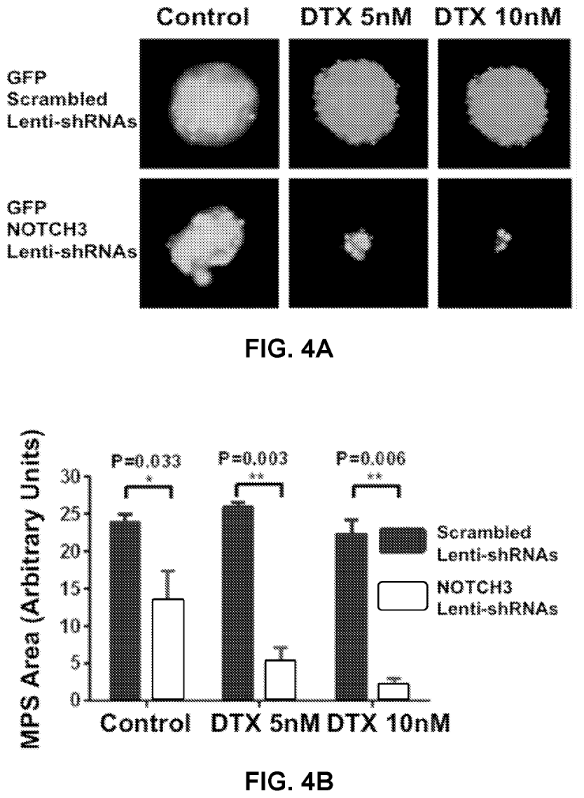

NOTCH3 genetic targeting reduces ALDH activity and enhances chemosensitivity: The extent to which NOTCH3 expression was required to induce the enrichment of ALDH^{high} TNBC cells that are responsible for high self-renewal capacity and intrinsic chemoresistance was investigated. TNBC-M40 3D-MPS were infected with NOTCH3 lenti-shRNAs or scrambled lenti-shRNAs used as control (FIG. 3A). NOTCH3 genetic targeting resulted in a reduction in ALDH activity, as compared to ALDH activity in TNBC-M40 3D-MPS infected with scrambled Lenti-shRNAs (FIG. 3B). To define the role of NOTCH3 expression in inducing self-renewal capacity and resistance to standard chemotherapy, TNBC-M25 3D-MPS (which exhibited the highest ALDH activity; FIG. 2B) were treated with docetaxel (DTX). DTX treatment did not inhibit 3D MPS growth, demonstrating that TNBCM25 3D-MPS exhibited intrinsic resistance to DTX-based chemotherapy (FIGS. 4A and 4B). However, NOTCH3 genetic targeting impaired 3D-MPS growth and enhanced sensitivity to DTX (FIGS. 4A and 4B).

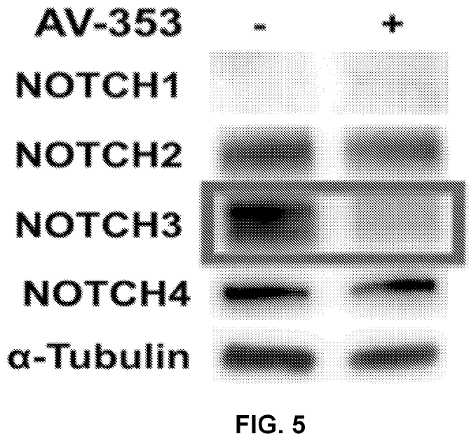

Humanized AV-353 antibody selectively targets NOTCH3 in TNBC cells: To assess the selectivity of a novel humanized antibody targeting NOTCH3 (AV-353), SUM149-PT TNBC cells expressing high endogenous levels of NOTCH3 were treated with AV-353 for 48 hours. As shown in FIG. 5, AV-353 (IC50: 200 ng) treatment reduced the level of NOTCH3-ICD, while NOTCH2-ICD and NOTCH4-ICD levels remained unchanged. NOTCH1-ICD was not expressed in SUM149-PT cells (FIG. 5).

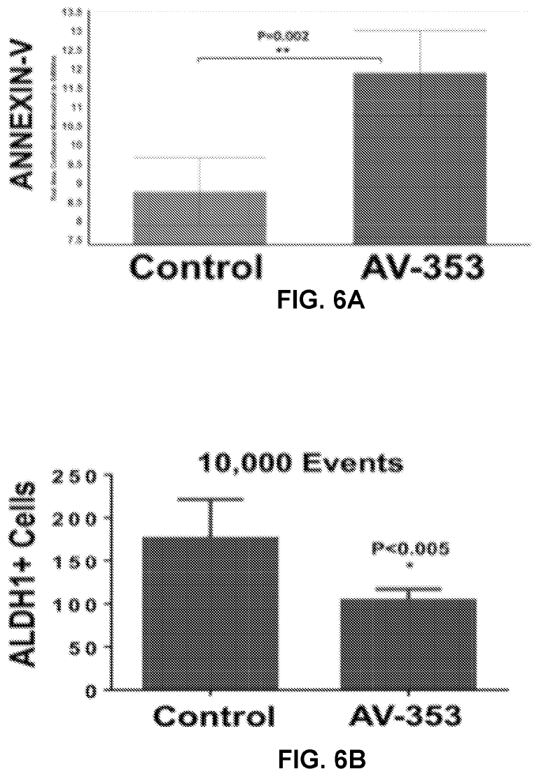

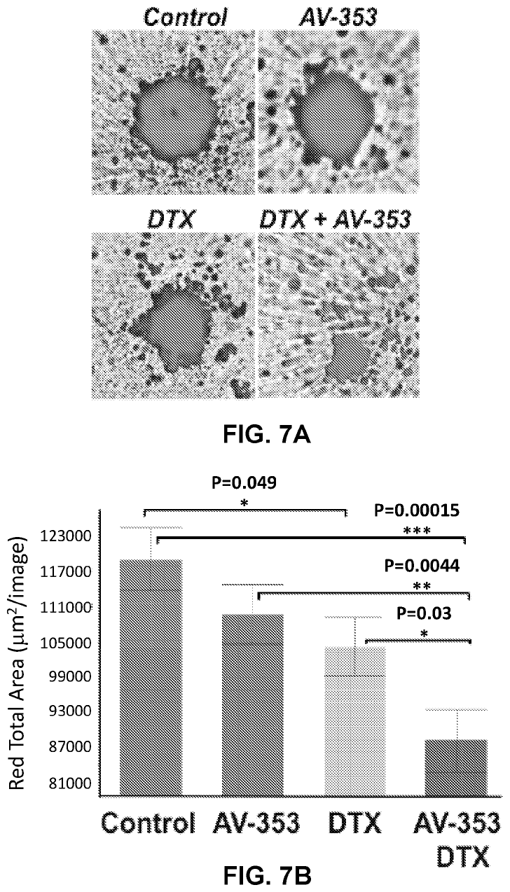

AV-353 treatment induces apoptosis and impairs ALDH activity and enhanced chemosensitivity: To evaluate the effect of AV-353 treatment in inducing apoptosis, SUM149-PT TNBC cells were treated with AV-353 (IC50: 200 ng) for 48 hours, and a real-time assay was performed using Annexin-V as a marker of early apoptosis. AV-353 treatment resulted in increased apoptosis in SUM149-PT cells (FIG. 6A). To define the extent to which AV-353-induced apoptosis was linked to inhibition of ALDH activity, SUM149-PT cells were treated with AV-353 (IC50: 200 ng), and an ALDEFLUOR™ assay (to detect ALDH^{High} cells) was performed after 48 hours. AV-353 resulted in a reduction in ALDH activity (FIG. 6B). AV-353 treatment also enhanced DTX sensitivity in 3D-MPS derived from SUM149-PT cells (FIGS. 7A and 7B). Taken together, these results demonstrated that AV-353 mimicked the effect of NOTCH3 genetic targeting (FIGS. 3A, 3B, 4A, and 4B) in TNBC cells.

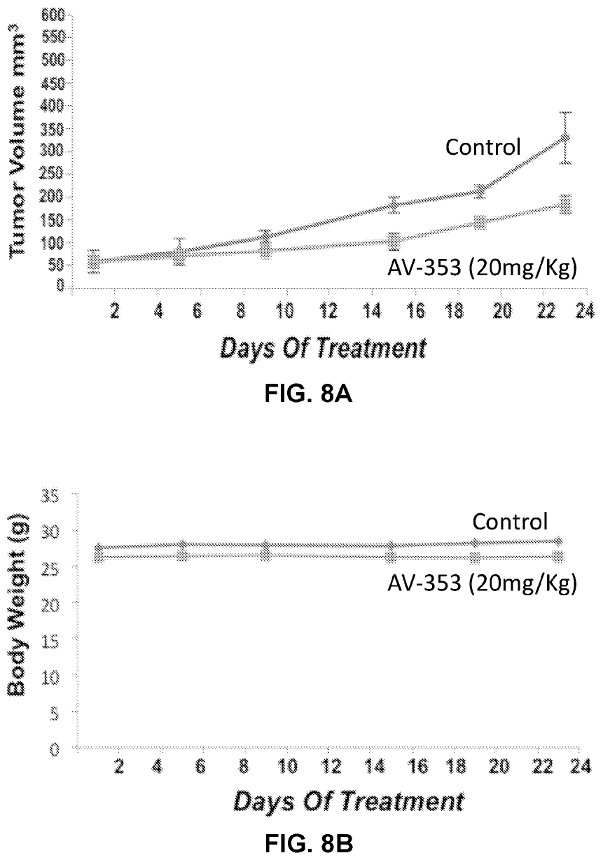

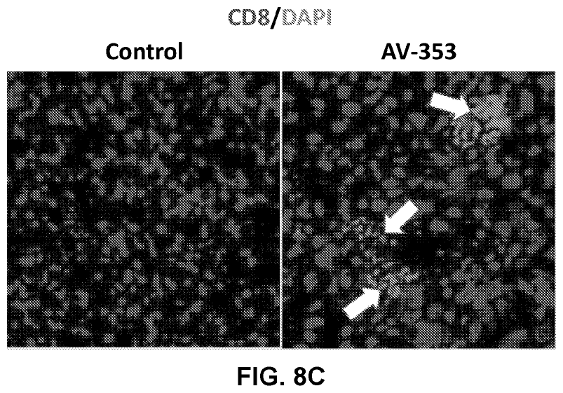

AV-353 inhibits the growth and impairs the immune evasion capacity of highly metastatic MDA-MB 231LM TNBC xenografts: MDA-MB 231/LM cells (isolated from lung metastasis) showed higher expression of NOTCH3 than parental MDA-MB 231 cells, as described elsewhere (Jalalirad et al., supra; and Leontovich et al., Breast Cancer Res. 2018, 20(1):105). MDA-MB 231/LM xenografts show an aggressive phenotype characterized by high organ metastatic burden (Jalalirad et al., supra). Treatment with AV-353 (20 mg/Kg) was tolerated in female humanized NSG-CD34⁺ mice, but reduced growth of MDA-MB 231/LM xenografts (FIGS. 8A and 8B) and also impaired the immune evasion capacity of the xenografts (FIG. 8C).

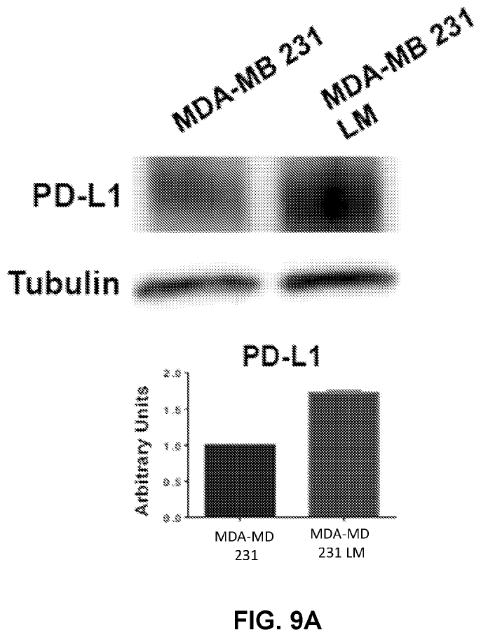

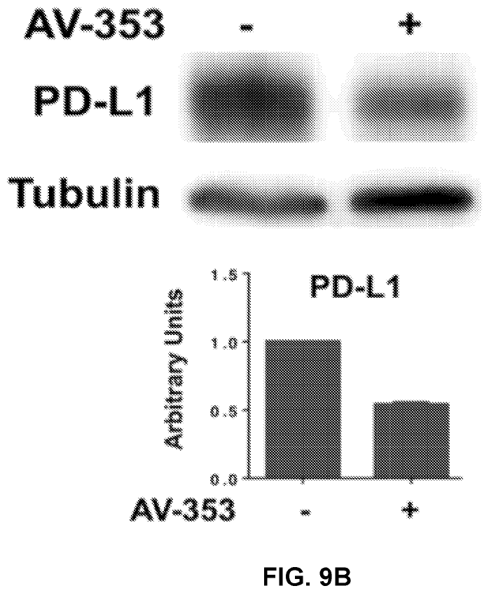

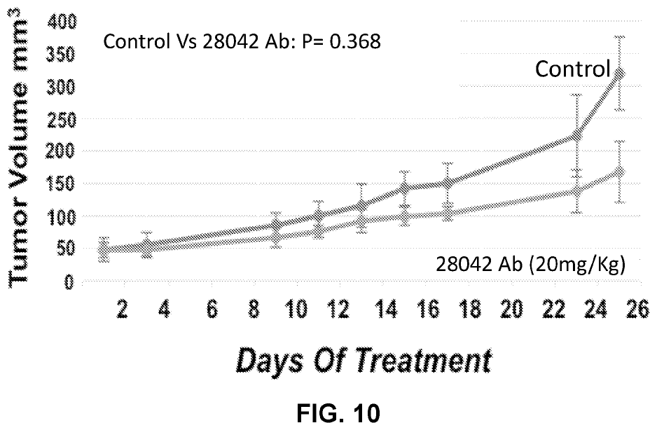

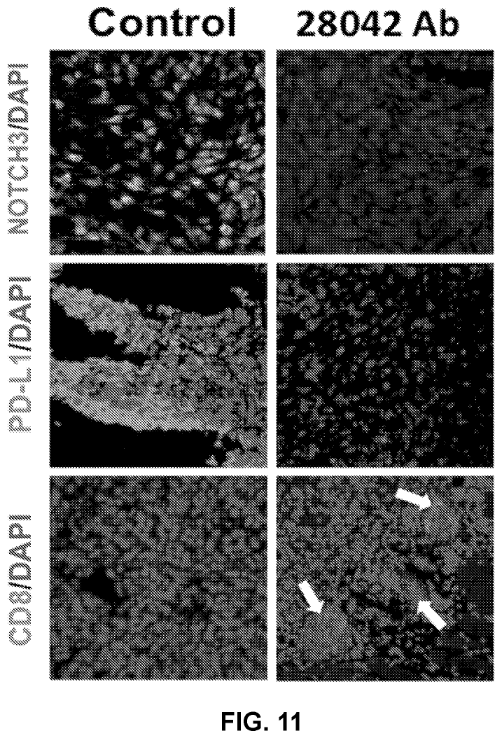

NOTCH3 pharmacologic targeting reduces PD-L1 expression and impairs the immune evasion capacity of TNBC cells: MDA-MB 231/LM cells expressed higher endogenous levels of PD-L1 trans-membrane protein compared to parental MDA-MB 231 cells (FIG. 9A). To define the role of NOTCH3 in promoting PD-L1 expression, MDA-MB 231/LM cells were treated with AV-353 for 48 hours. AV-353 treatment reduced PD-L1 expression (FIG. 9B), demonstrating a link between NOTCH3 signaling and immune checkpoint regulation. To evaluate the therapeutic efficacy of NOTCH3 targeting in reducing PD-L1 expression and impairing the immune evasion capacity of TNBC cells, TNBC-4T1 syngeneic tumors that showed high levels of endogenous NOTCH3 and PD-L1 were established (FIG. 11). Treatment of TNBC-4T1 syngeneic tumors with 28042 (20 mg/Kg), a non-humanized selective anti-NOTCH3 antibody (AVEO Oncology; Boston, MA) reduced tumor growth (FIG. 10). Treatment with 28042 also reduced NOTCH3 and PD-L1 expression, and induced tumor infiltration of CD8+ cytotoxic T cells (FIG. 11). Taken together, these studies demonstrated that NOTCH3 pharmacologic targeting reduces PD-L1 expression and inhibits the immune evasion capacity of TNBC cells.

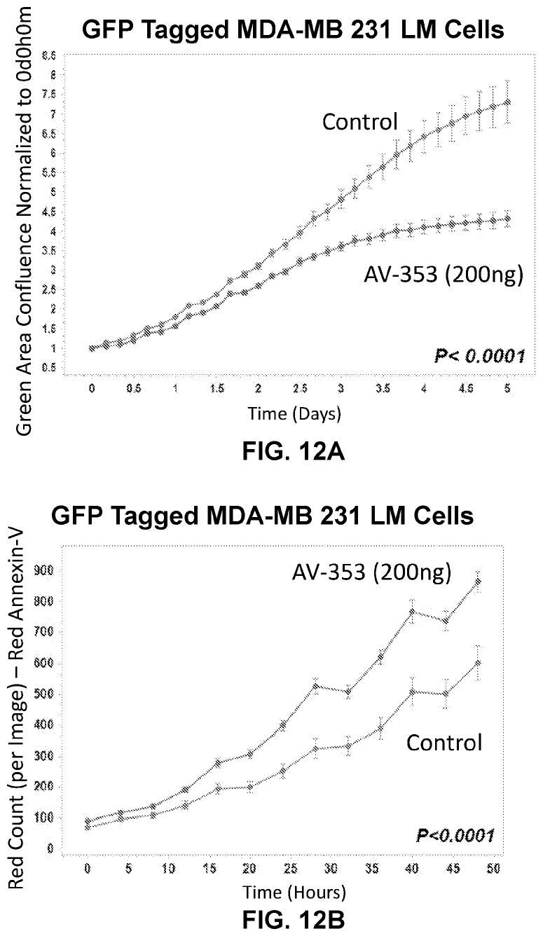

AV-353 impaired cell proliferation and induced apoptosis in TNBC cells: GFP-tagged MDA-MB 231 LM cells were assessed for real-time cell proliferation and apoptosis (using Annexin-V as a marker), before and after treatment with AV-353 (IC50: 200 ng) for 5 days. Cell proliferation and apoptosis were quantified using the INCUCYTE® Cell Player System (Sartorius; Goettingen, Germany). Three independent experiments were performed in triplicate (±S.D.). These studies demonstrated that AV-353 impaired cell proliferation and induced apoptosis in TNBC cells (FIGS. 12A and 12B).

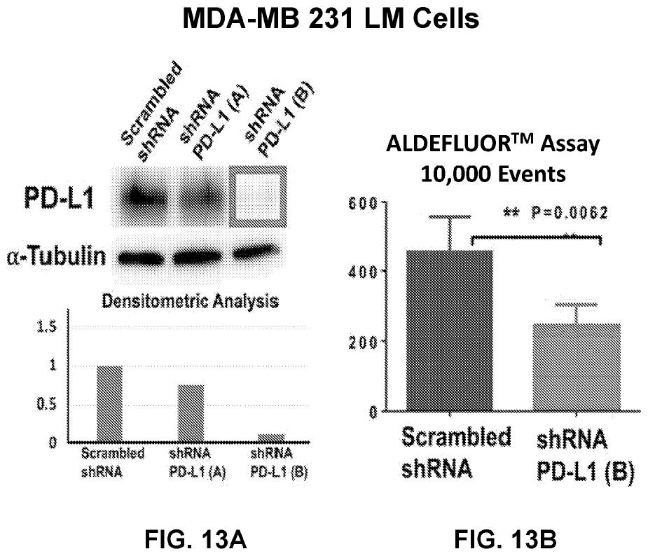

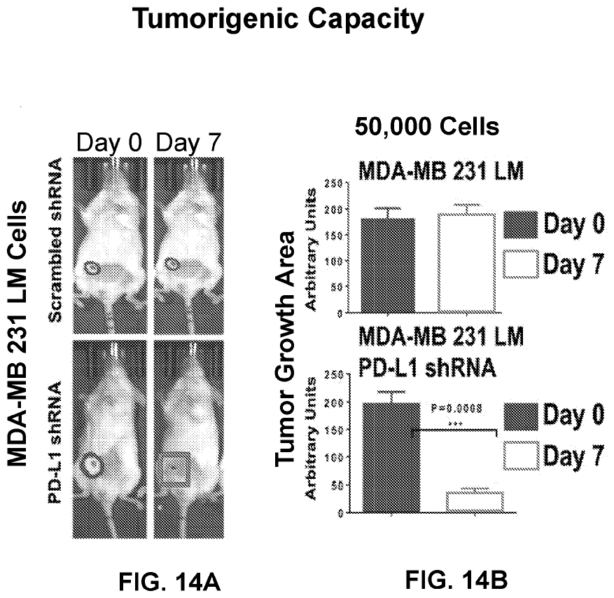

Tumor cell-intrinsic PD-L1 oncogenic signaling pathway: To define the pivotal role of tumor cell-intrinsic PD-L1 expression in inducing enrichment of ALDH^{high} CSCs with high tumorigenic capacity, MDA-MB 231/LM cells were infected with scrambled shRNAs or PD-L1 shRNAs. PD-L1 genetic targeting reduced the enrichment of ALDH^{high} CSCs (FIGS. 13A and 13B). To define the role of PD-L1 expression in vivo, 50,000 MDA-MB 231/LM cells infected with scrambled shRNAs or PD-L1 shRNAs (expressing luciferase lentivectors) were injected into the mammary fat pad of female NSG mice, and tumor growth was monitored using the Xenogen instrument. PD-L1 genetic targeting impaired in vivo tumorigenic capacity of MDA-MB 231/LM cells (FIGS. 14A and 14B).

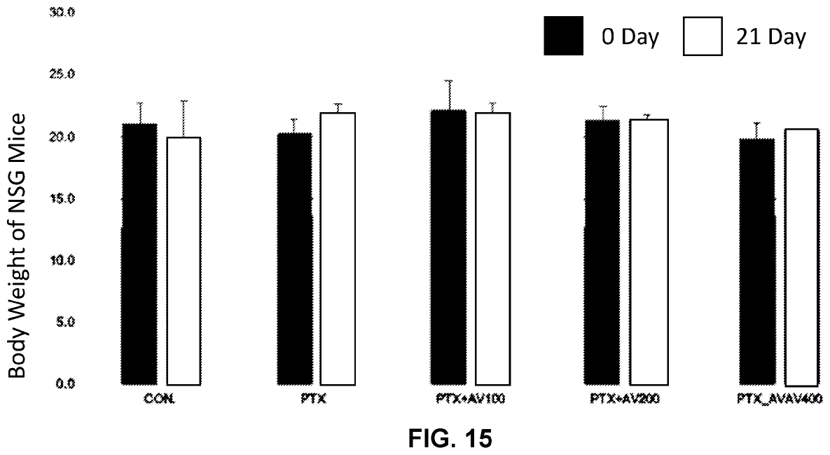

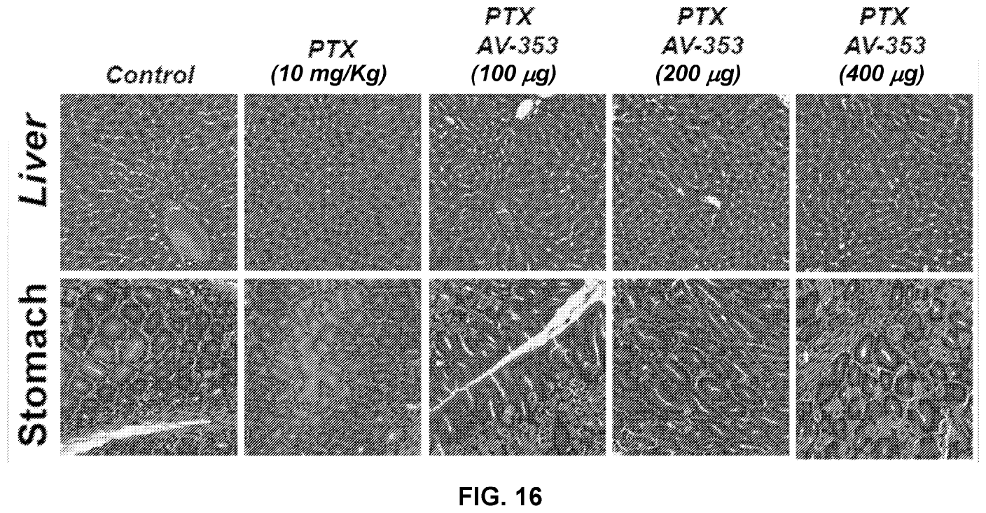

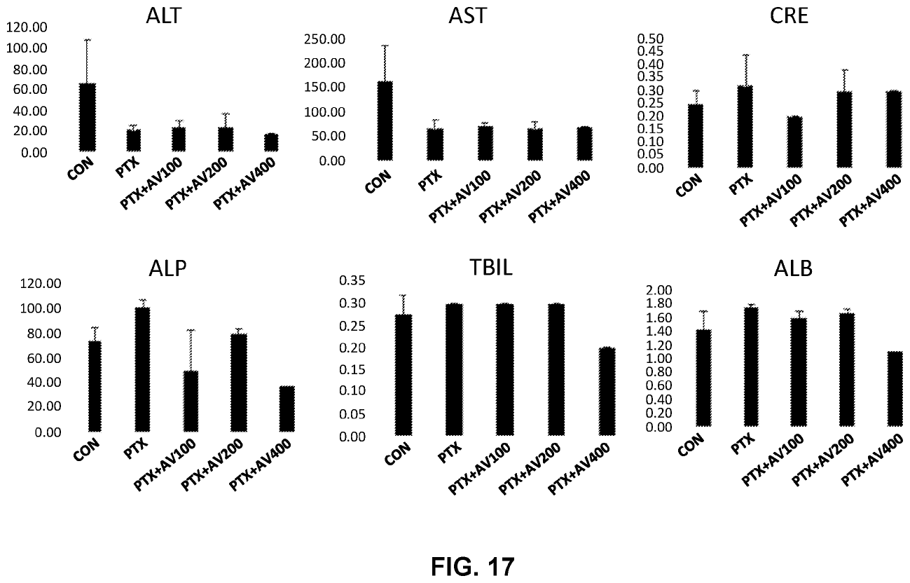

Toxicology studies: Female NSG mice received saline solution (control) or 10 mg/Kg paclitaxel (PTX) as monotherapy or in combination with escalating doses of AV-353 (AV100 ug, AV200 ug, and AV400 ug). PTX and AV-353 were administered intraperitoneally 3 times per week for 4 weeks. PTX as monotherapy and in combination with AV-353 did not induce body weight loss (FIG. 15), indicating that this combination was well-tolerated in vivo. After drug treatment, animals were euthanized and organs of interest (liver and stomach) were paraffin embedded, sectioned, and hematoxylin and eosin stained. No significant damage was detected in stomach or liver tissue collected from animals that received PTX as monotherapy or in combination with escalating doses of AV-353 (FIG. 16). Although no liver damage was detected at the morphological or histological level, further studies were conducted to determine the extent to which the drug treatments induced an increase of liver enzymes. Blood samples were collected from animals that received saline solution (control) and the various drug treatments, and liver enzyme analysis was executed using the VETSCAN® instrument (Zoetis) to measure levels of ALT, AST, ALP, TBIL, CRE, and ALB (FIG. 17). These studies demonstrated that none of the drug treatments induced a significant increase in the liver enzymes evaluated.

### Example 2—NOTCH3 Blockade Inhibits Cancer Cell Plasticity Through a NOTCH3/PD-L1 Oncogenic Axis in Triple Negative Breast Cancer

The results in this Example re-present and expand on at least some of the results provided in the other Example.

### Materials and Methods

Human Breast Cancer Cell Lines: The human breast cancer cell line MDA-MB 231 was obtained from ATCC (Manassas, VA). SUM149-PT breast cancer cells were obtained from the Couch laboratory (Mayo Clinic). All cell lines were maintained in DMEM medium containing 5 mM glutamine, 1% penicillin/streptomycin and 10% FBS at 37° C. in 5% CO₂ atmosphere. PDX-derived TNBC cells (M14, M25, and M40) were established from metastatic PDX models in the Sarkaria laboratory (Mayo Clinic Cancer Center).

Mammosphere Formation: Human breast cancer cells were plated in ultra-low attachment 24- and 96-well culture dishes in 100 μL of MAMMOCULT™ medium (STEMCELL™ Technologies), and medium was added every 2 days for a maximum of 8 days. Mammospheres were trypsinized and expanded every 8 days to form tertiary mammospheres, and their growth was recorded through a digital camera (Nikon).

Real-Time Cell Proliferation, Cytotoxicity and Apoptosis Assays: 5,000 cells were plated in Costar 94-well plates, treated with AV-353 (AVEO Oncology), Docetaxel or a combination thereof. Cell proliferation and apoptotic cells were quantified in real-time using INCUCYTE® S3 (BioEssen). Experiments were performed in triplicate (±S.D.).

Immunoblot, Immunofluorescence and LACS Assays: Immunofluorescence and FACS assays were performed as described elsewhere (D'Assoro et al., supra).

Lenti-vectors Targeting NOTCH3 and PD-L1: Scrambled (control) and GFP-tagged NOTCH3 and PD-L1 shRNA lenti-vectors were obtained from OriGene Technologies (Rockville, MD) and used according to the manufacturer's instructions.

ALDH Activity Assay: ALDH activity was detected by FACS analysis using the ALDEFLUOR® assay kit (STEMCELL™ Technologies) as described elsewhere (Jalalirad et al., supra). Results were derived from three independent experiments with comparable outcomes (±S.D.).

shRNAs: shRNAs were obtained from Integrated DNA Technologies (IDT; Coralville, IA), and included the following sequences:

(SEQ ID NO: 3)
GGACAUGCAGGAUAGCAAGGAGGG,
(SEQ ID NO: 4)
AGAUUAAUGAGGAUGACUGCGGCCC,
and
(SEQ ID NO: 5)
AGAUGGGACAUGUUCCAUAGCCUTG,
as well as a universal
scrambled control (cat. no. SR30004, IDT).

RNA_Seq data Analysis: RNA sequencing fastq data was processed using the Mayo Analysis Pipeline for RNA sequencing (MAP-RSeq, version 3.1.1) (Kalari et al., BMC Bioinformatics 2014, 15:224). This pipeline includes read alignment, quality control, gene expression quantification, and gene-fusion identification. The alignment of RNA-Seq reads was performed using the STAR aligner (version 2.5.2b) (Dobin et al., Bioinformatics 2013, 29(1):15-21). The reference genome used for this analysis was GRCh38 (hg38). Gene expression quantification was performed using the Subread package (version 1.5.1) to obtain both raw and FPKM counts (Liao et al., Nucleic Acids Res 2013, 41(10):e108).

Mayo TNBC cohort and Claudin-low subtype identification: A group of women who were diagnosed with TNBC and who received upfront surgery (without neoadjuvant therapy) were selected for this study. The process of gathering the cohort and their clinical and pathological characteristics and outcomes are described elsewhere (Leon-Ferre et al., Breast Cancer Research and Treatment 2018, 167:89-99). Of the 605 women in the cohort, formalin-fixed paraffin-embedded primary breast tissue was available for RNA extraction and sequencing in 269 cases (see, Thompson et al., NAR Cancer 2022, 4(2):zcac018). RNA-Seq data from the 269 tumors was used to identify the presence of Claudin-low TNBC tumors using a machine learning classification predictor from the R package “genefu.” This predictor utilized median-centered RPKM values and 807 gene signatures from a study described elsewhere (Prat et al., Breast Cancer Res 2010, 12:R68), which was based on nine claudin-low cell lines (BT549, HBL100, HS578T, MDAMB157, MDAMB231, MDAMB435, MDAMB436, SUM159PT, SUM1315).

Differential Expression Analysis and Pathway Enrichment Analysis: Differential expression (DE) analysis was performed using the empirical Bayes quasi-likelihood F-test (QLF) implemented in the R package edgeR. Genes were considered significantly differentially expressed if their absolute fold change was >2 and their p-value was <0.05. The R package ReactomePA was then employed to conduct a hypergeometric test to identify the enriched pathways within the gene set of interest, which were subsequently visualized using a cnetplot created using clusterProfiler.

Breast Tumor Xenografts: Breast tumor xenografts were established as described elsewhere (Jalalirad et al., supra).

Scientific Rigor and Statistical Analysis: Experiments were run in triplicate or for 3 independent runs (±S.D.). The average read-out of the triplicates from each run was determined and a 95% t-confidence interval for the difference between was constructed. Different breast cancer cell lines were employed in this study to increase the power to detect the effect size. The nonparametric Mann-Whitney t-test (Statview software) was used to determine the significance of the relative tumor volumes for treated versus untreated tumor xenograft groups. Initially, a sample size of 5 animals per group was utilized, but this was increased to 10 animals per group if experimental outcomes warranted. A two-sided (alpha=0.05), two-sample t-test was then conducted to determine whether the mean tumor burden differed significantly between a particular pair of treatment groups. This test had a power of 90% to detect a difference of 1.6 standard deviations (SD). For each xenograft (treated and control groups), the difference in the percentage of organ metastatic burden was assessed. Animals were examined daily and body weight and primary tumor size were measured three times per week. Survival analysis was performed using the survival R package (Therneau and Grambsch, Modeling Survival Data: Extending the Cox Model, Springer, New York, 2000) and Kaplan-Meier plots were generated using survminer (Kassambara, survminer: Drawing Survival Curves using ‘ggplot2’, 2017; available online at cran.r-project.org/web/packages/survminer/index.html). Optimal cut points were selected for dichotomizing the sample cohort for each gene using the maximally selected rank statistics (Hothorn and Lausen, Computational Statistics & Data Analysis 2003, 43(2):121-137) considering the middle 80% of the distribution. A Cox proportional hazard regression analysis was conducted to examine the impact of higher expression above the optimal cut point on the prognostic outcomes.

### Results

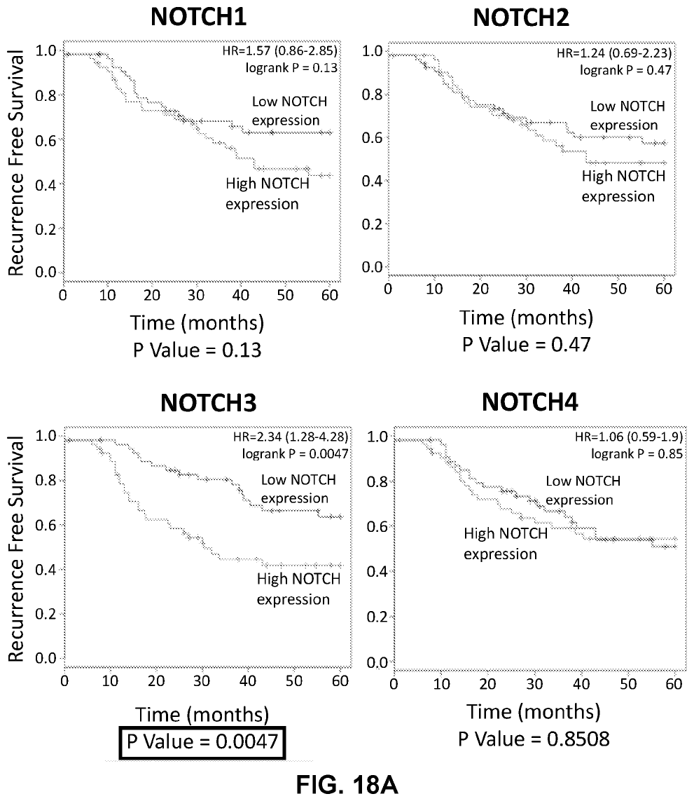

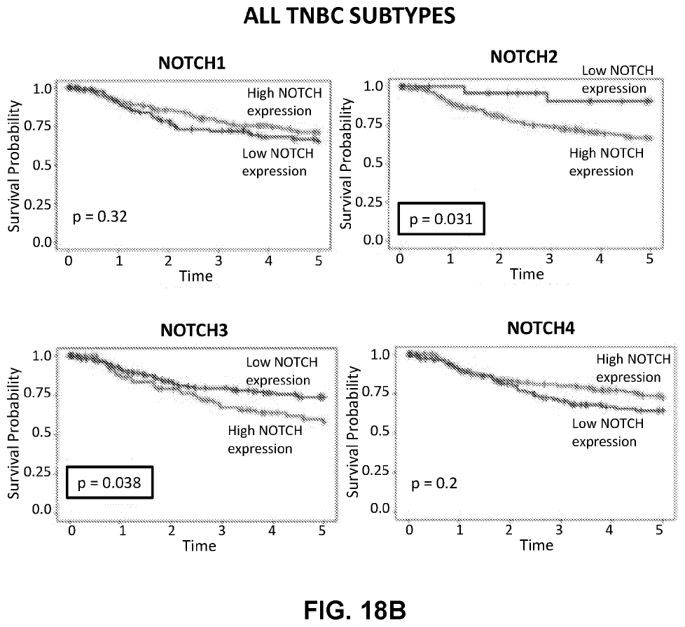

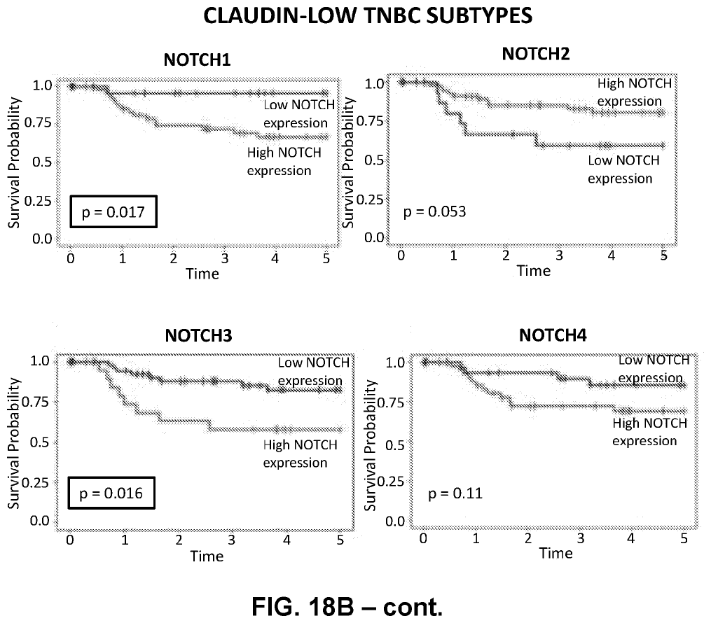

High NOTCH3 mRNA Expression is Linked to Poor Outcome of TNBC Patients: To address which of the different NOTCH receptors may be linked to shorter recurrence free survival (RFS), a selected cohort of 107 patients with lymph node⁺ TNBC was analyzed. Amongst the four NOTCH receptors, only high NOTCH3 mRNA expression was significantly linked to reduced RFS (FIG. 18A), indicating that NOTCH3 expression may drive TNBC progression. The findings were confirmed by analyzing a separate TNBC gene expression dataset consisting of 269 patients from the Mayo Clinic cohort. Additionally, a claudin-low subtype was identified from this dataset (N=91 patients), the expression of NOTCH genes in this subgroup was studied. The prognostic value of NOTCH genes using the five-year distant disease-free survival (DDFS) data from both the Mayo Clinic RNA-Seq cohort and the claudin-low cohort was evaluated. The result of the evaluation suggested that, among the four NOTCH genes, higher expression of NOTCH2 and NOTCH3 genes was marginally associated with worse DDFS outcomes (NOTCH2: HR 4.51, 95% CI 1.02-16.96, p-value=0.048; NOTCH3: HR 1.67, 95% CI 1.02-2.73, p-value=0.041) (FIG. 18B). Moreover, higher expression of NOTCH3 (HR 3.14, 95% CI 1.17-8.38, p-value=0.02) was also associated with poor DDFS outcomes in claudin-low patients (FIG. 18B).

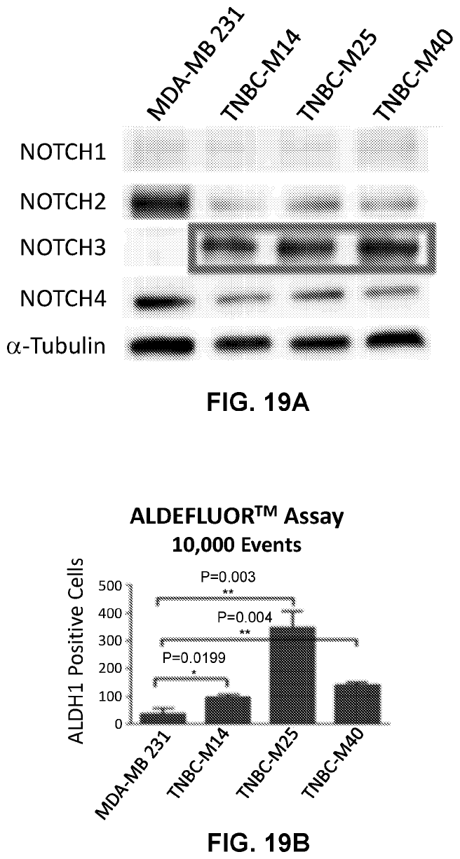

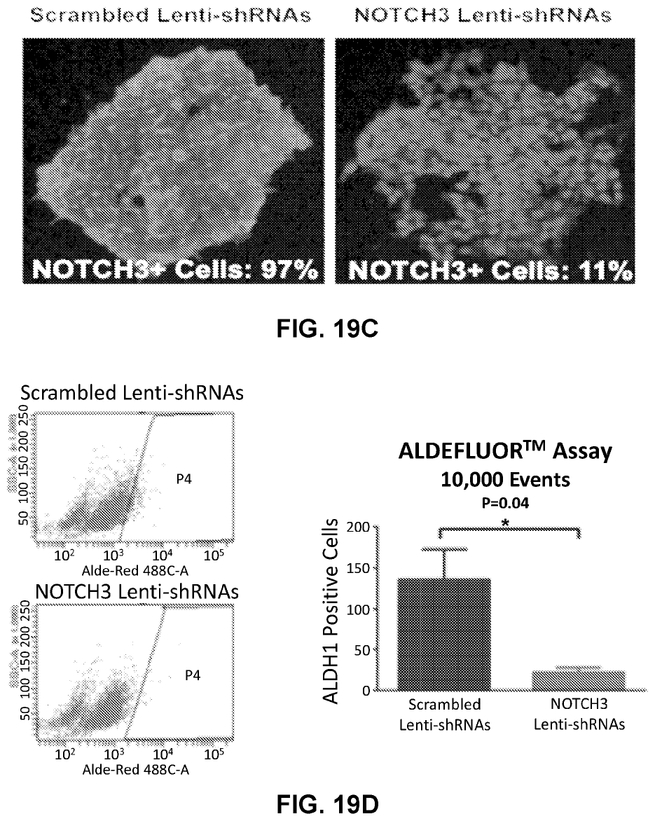

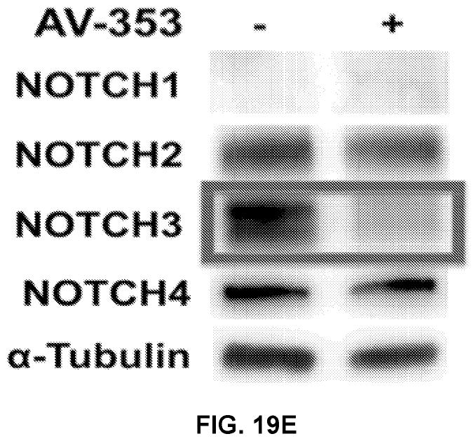

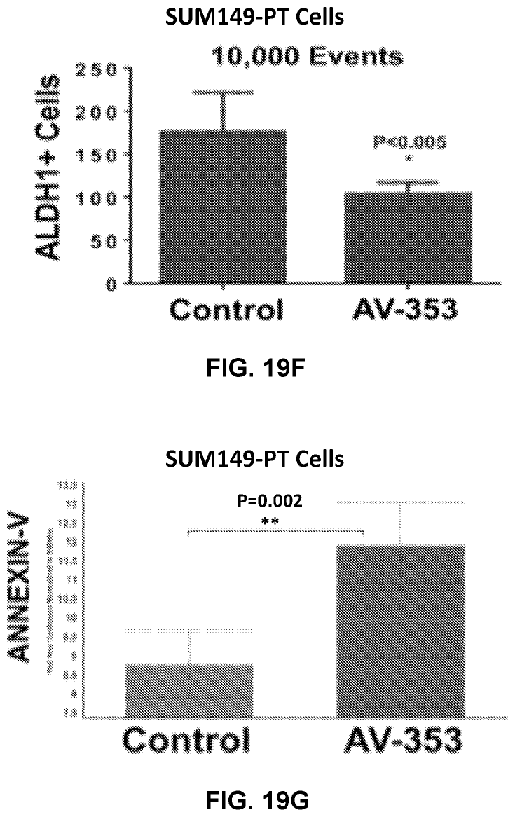

NOTCH3 Expression is Necessary for the Enrichment of ALDH^{high} Cancer Stem Cells: Breast cancer cells that undergo epithelial-to-mesenchymal (EMT)-mediated cancer plasticity acquire a CD44^{high}/CD24t° and/or ALDH^{high} cancer stem-like cell phenotype that confers high self-renewal capacity (Mani et al., Cell 2008, 133(4):704-715; Xu et al., Biochem Biophys Res Commun 2018 502(1):160-165; Opyrchal et al., Oncotarget 2017, 8(53):91803-91816; Jalalirad et al., supra; and D'Assoro et al., supra). High ALDH activity can induce intrinsic drug resistance following treatment with standard of care chemotherapy (Tomita et al., Oncotarget 2016, 7(10):11018-11032; Toledo-Guzmán et al., Curr Stem Cell Res Ther 2019, 14(5):375-388; and Huddle et al., J Med Chem 2018, 61(19):8754-8773) and can contribute to the emergence of organ metastasis. To investigate a mechanistic link between NOTCH3 and ALDH, the extent to which NOTCH3 expression correlated with an ALDH^{high} phenotype was explored in two different cell line systems. First, ex-vivo 3D-Mammospheres (MPS) were established using unique TNBC cells (TNBC-M14, TNBC-M25 and TNBC-M40) isolated from metastatic patient-derived xenografts (PDXs) (Jalalirad et al., supra; and Leontovich et al., supra). TNBC-M14, TNBC-M25 and TNBC-M40 3D-MPS showed NOTCH3 overexpression as compared to MDA-MB 231 3D-MPS, which was used as a control (FIG. 19A), indicating that NOTCH3 protein levels increase during tumor progression. The higher NOTCH3 protein levels in TNBC-M14, TNBC-M25 and TNBC-M40 3D-MPS correlated with higher ALDH activity compared to MDA-MB 231 parental 3D-MPS (FIG. 19B).

Next, the effect of blocking NOTCH3 expression was investigated. TNBC-M40 3D-MPS cells were infected with NOTCH3 lentivirus-mediated shRNA or scrambled shRNA as a control. These studies demonstrated that the proportion of NOTCH3-positive cells dropped from 97 to 11% following introduction of NOTCH3 shRNA (FIG. 19C). The loss of NOTCH3 expression was accompanied by a significant reduction of ALDH activity compared to TNBC-M40 3D-MPS infected with scrambled shRNA (FIG. 19D).

The relationship between NOTCH3 signaling and ALDH activity was then evaluated in a different cell line model and using a different mode of Notch signaling blockade. Specifically, the SUM149-PT cell line, which is derived from a mouse xenografted with a ductal carcinoma metastatic nodule (Jalalirad et al., supra; and D'Assoro et al., supra) was used, and NOTCH3 signaling was abrogated by a novel humanized antibody (AV-353) that blocks NOTCH3 proteolytic processing and thus downstream activation and NOTCH3-mediated nuclear reprogramming. SUM149-PT cells express high endogenous levels of NOTCH3, and AV-353 treatment reduced the levels of NOTCH3 ICD, while the levels of NOTCH2 ICD and NOTCH4 ICD remained unchanged and NOTCH1 ICD was not expressed in the SUM149-PT cells (FIG. 19E). To determine whether selective NOTCH3 pharmacologic targeting also reduced the enrichment of ALDH^{high} CSCs, SUM149-PT cells were treated with AV-353 and the ALDEFLUOR™ assay was performed after 48 hours. AV-353 induced a significant reduction of ALDH activity (FIG. 19F). To assess the effect of AV-353 treatment in inducing apoptosis, the level of Annexin-V was monitored as a marker of early apoptosis following AV-353 for 48 hours in the SUM149-PT cells. AV-353 treatment significantly increased apoptosis in SUM149-PT cells (FIG. 19G). Taken together, these studies suggested that NOTCH3 enhances the enrichment of ALDH^{high} CSCs and that NOTCH3 is a key driver of stemness in TNBC cells.

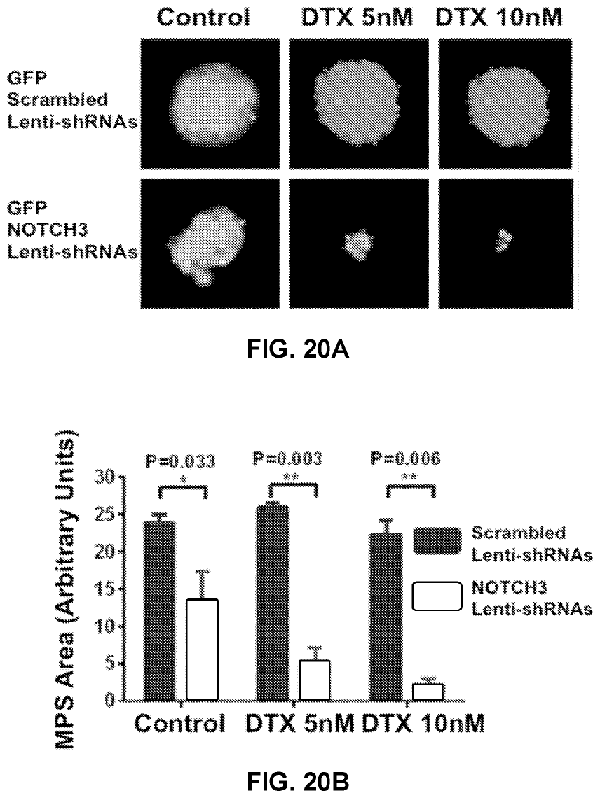

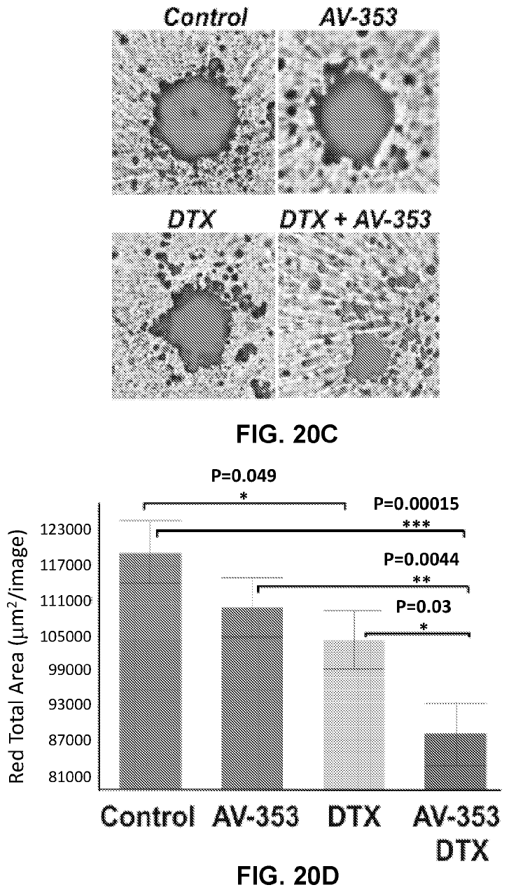

NOTCH3 Blockade Enhances Chemosensitivity in TNBC Cells: To explore whether NOTCH3 function is pivotal to induce chemoresistance in TNBC cells, TNBC-M25 3D-MPS cells (which showed the highest ALDH activity; FIG. 19B) stably expressing NOTCH3 or scrambled shRNA were treated with docetaxel (DTX) at different dosages. NOTCH3 shRNA TNBC-M25 3D-MPS grew somewhat slower than the scrambled shRNA control line, and upon addition of 5 and 10 nM DTX, growth was severely compromised in NOTCH3 shRNA TNBC-M25 3D-MPS, but not in the control line (FIGS. 20A and 20B). Further studies were conducted to assess whether NOTCH3 pharmacologic blockade enhanced chemosensitivity in the SUM-149 PT cells. Similarly to TNBC-M25 3D-MPS, AV-353 treatment alone did not significantly reduce mammosphere growth (FIGS. 20C and 20D). DTX treatment at 5 nM had a small effect on growth, while the combination of AV-353 and DTX treatment strongly reduced growth (FIGS. 20C and 20D). Thus, these studies demonstrated that NOTCH3 blockade enhances chemosensitivity in TNBC cells.

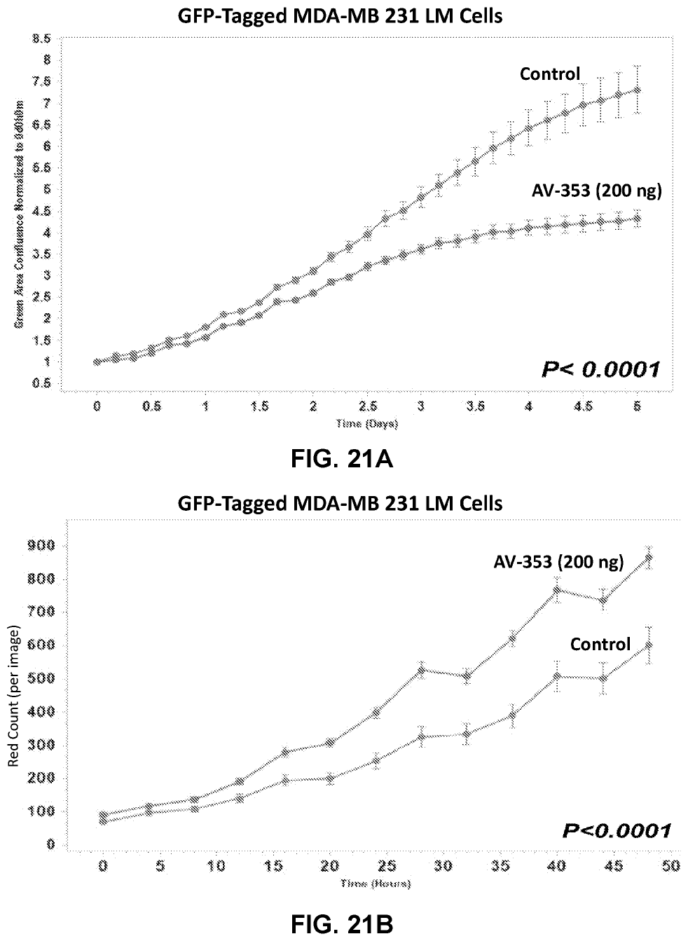

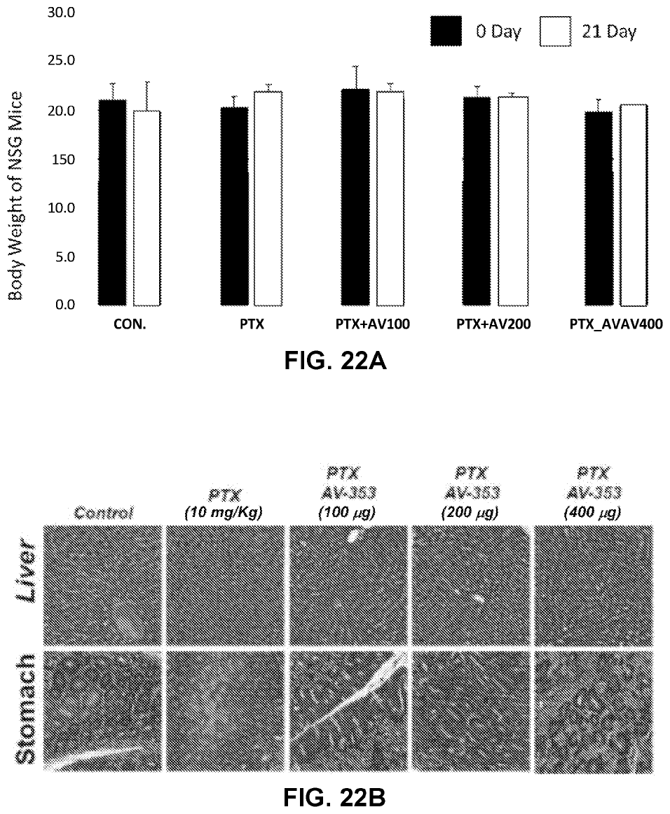

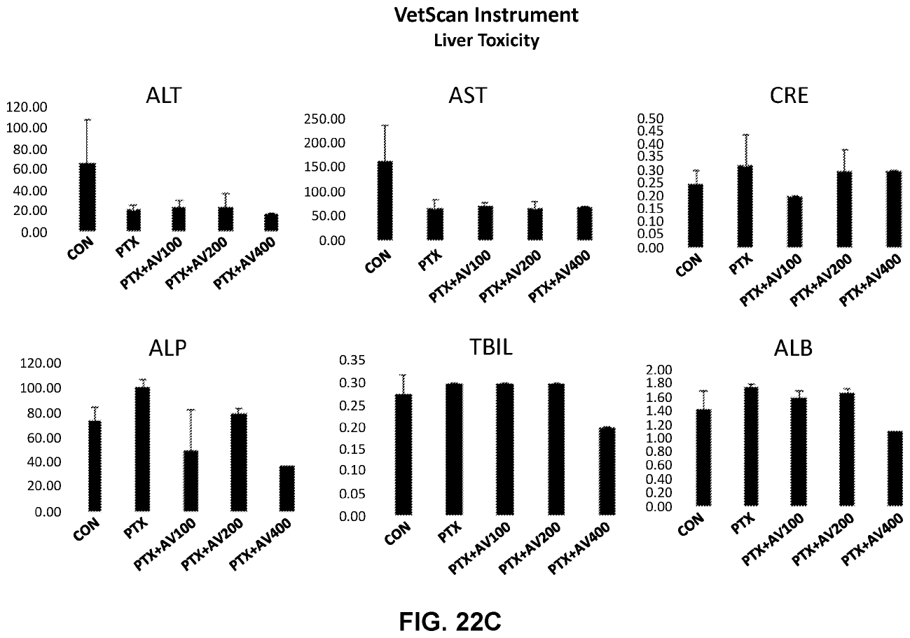

NOTCH3 Pharmacologic Blockade Inhibits Tumor Growth of Xenografted TNBC Cells: To determine the effect of NOTCH3 blockade on TNBC tumor growth in vivo, the MDA-MB 231/LM cell line was used. MDA-MB 231/LM was isolated from lung metastasis, shows a more aggressive phenotype after xenografting in mice, and expresses higher levels of NOTCH3 compared to parental MDA-MB 231 cells (Jalalirid et al., supra; and Leontovich et al., supra). These studies demonstrated that in vitro treatment of MDA-MB 231/LM cells impaired cell proliferation and induced apoptosis (FIGS. 21A and 21B). Significantly, dose-escalation studies with AV-353 in combination with Paclitaxel (PTX) using healthy NSG mice demonstrated that AV-353 was well tolerated in animals which showed normal body weight and no significant GI toxicity (FIGS. 22A and 22B). Blood samples were collected from animals that received saline solution (control) and the various drug treatments, and liver enzyme analysis was executed using the VETSCAN® instrument (Zoetis) to measure levels of ALT, AST, ALP, TBIL, CRE, and ALB (FIG. 22C). These studies demonstrated that none of the drug treatments induced a significant increase in the liver enzymes evaluated.

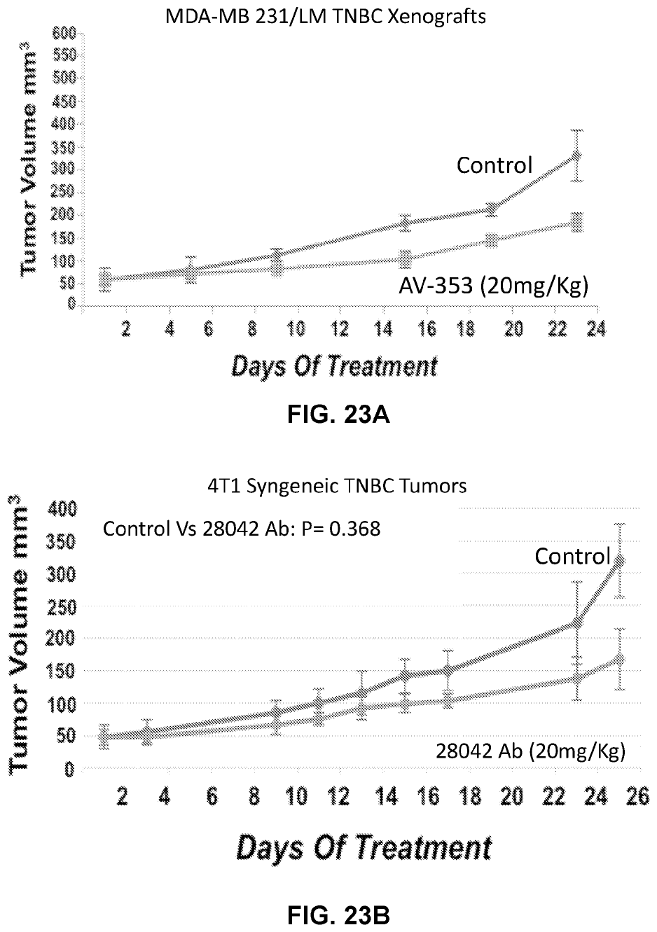

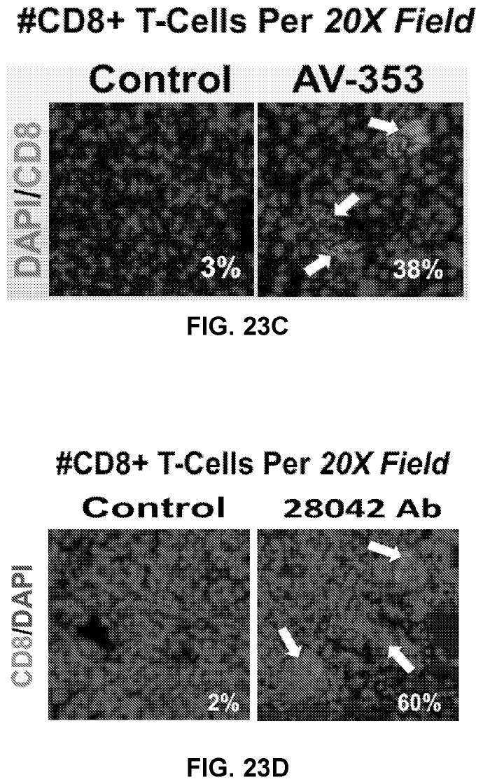

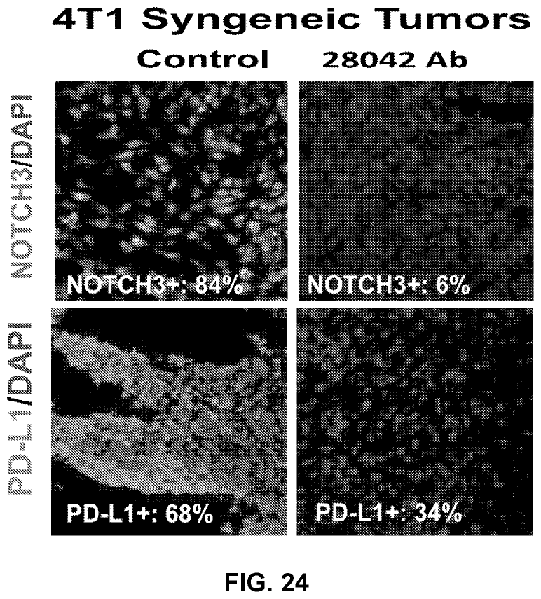

AV-353 treatment as monotherapy reduced MDA-MB 231/LM xenograft growth in humanized NSG-CD34⁺ mice (FIG. 23A). To corroborate these findings, another NOTCH3 blocking antibody (28042; a non-humanized selective anti-NOTCH3 antibody) was used. TNBC-4T1 syngeneic tumors were established and mice were treated with 20 mg/Kg of 28042, resulting in a reduction in tumor growth (FIG. 23B) that was linked to reduced expression of NOTCH3 and PD-L1 (FIG. 24). Taken together, these data indicated that NOTCH3 blockade by two different blocking antibodies reduces tumor growth in vivo.

NOTCH3 Acts Epistatically Over the PD-L1 Signaling Pathway: The results discussed above demonstrated that blockade of NOTCH3 reduces ALDH activity and stemness, and also leads to reduced tumor growth in vivo. Further studies were conducted to determine how NOTCH3 exerts its tumor-promoting effects. In the two mouse models treated with NOTCH3 blocking antibodies, an increase in the number of infiltrating CD8-positive cytotoxic T-cells was observed (FIGS. 23C and 23D), suggesting that the immune regulation in the tumor microenvironment is altered by NOTCH3 blockade.

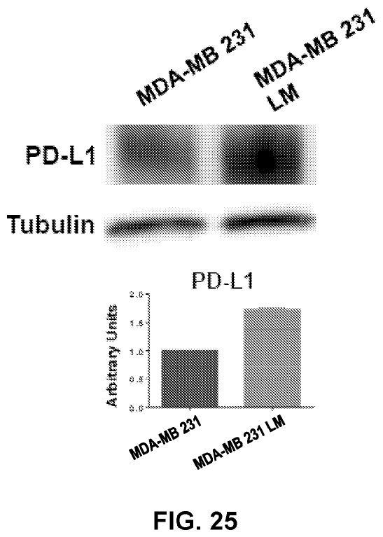

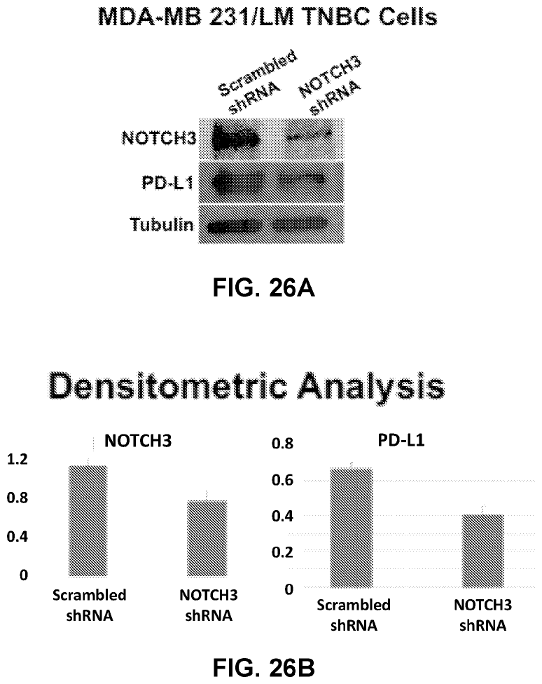

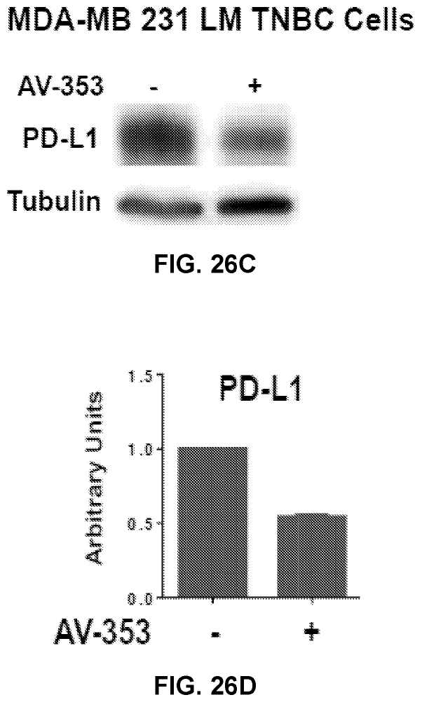

Thus, studies were conducted to explore whether NOTCH3 can regulate the PD-1/PD-L1 immune check point system. NOTCH3 expression was stably knocked down using NOTCH3 shRNA in the MDA-MB-231/LM cell line, which expressed higher levels of PD-L1 than the parental MDA-MB-231 cells (FIG. 25A). Knockdown of NOTCH3 protein levels in MDA-MB-231/LM cells led to a reduction in expression of the PD-L1 ligand (FIGS. 26A and 26B). To corroborate this observation, MDA-MB-231/LM cells were treated with AV-353 for 48 hours, which similarly reduced intra-tumoral PD-L1 expression (FIGS. 26C and 26D).

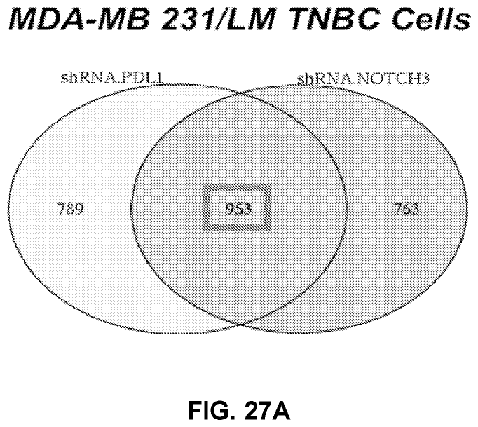

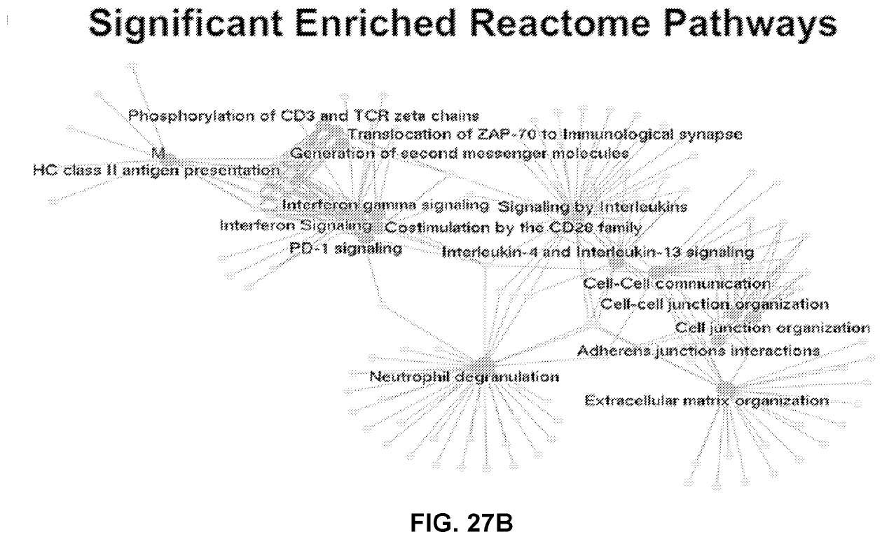

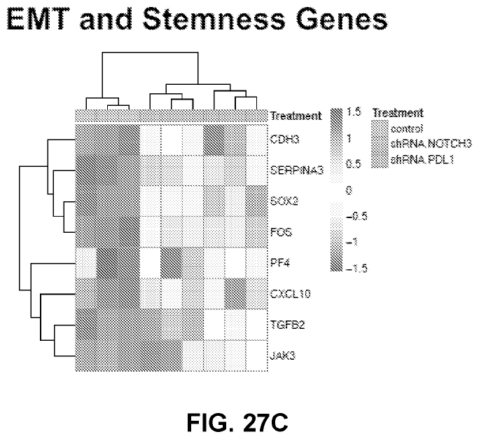

To gain insight into the mechanistic link between NOTCH3 and PD-L1 signaling pathways, a transcriptomic analysis was performed using MDA-MB-231/LM cells in which NOTCH3 or PD-L1 was stably knocked down by shRNA. Knockdown of PD-L1 and NOTCH3 resulted in about 50% and 20% reduced expression at the mRNA level, respectively. At the protein level, both NOTCH3 and PD-L1 knockdown were more efficient: NOTCH3 protein was reduced by about 50%, while PD-L1 protein was reduced by about 90%. In total, 1742 DEGs were identified from the PD-L1 vs control set and 1716 DEGs were identified from the NOTCH3 versus the control set. More than half of the genes (953 DEGs) were common between the two datasets (FIG. 27A). Among those 953 genes, 125 genes were identified that regulate EMT, immune evasion, stemness and metastasis (FIG. 27B and TABLE 1). NOTCH3 or PD-L1 genetic targeting induced a significant down-regulation of EMT and sternness genes (FIG. 27C) that are responsible for cancer cell plasticity and organ metastasis. Collectively, these data demonstrated that NOTCH3 acts upstream (epistatically) over PD-L1, and that a substantial fraction of genes involved in EMT-mediated cancer cell plasticity and sternness regulated by NOTCH3 are also regulated by PD-L1.

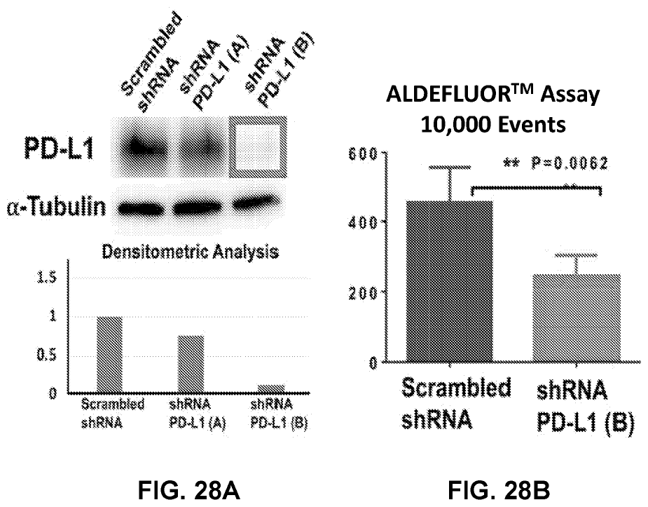

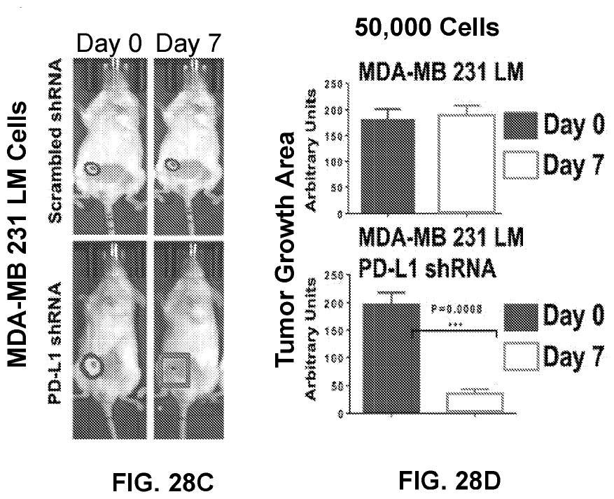

Reducing PD-L1 Expression Inhibits ALDH Activity and In Vivo Tumorigenic Capacity of MDA-MB 231 LM TNBC Cells: The data discussed above place NOTCH3 epistatically over PD-L1 and define intra-tumoral PD-L1 as an important component of the Notch downstream response. Since PD-L1 genetic targeting was shown to significantly reduce the expression of EMT and sternness genes (FIG. 27C), studies were conducted to explore whether intra-tumor cell reduction of PD-L1 was sufficient to evoke a tumor-inhibiting phenotype. Indeed, MDA-MB-231/LM cells stably knocked down for PD-L1 showed significantly reduced ALDH levels, as determined by ALDEFLUOR™ assay (FIGS. 28A and 28B). Finally, upon xenografting of PD-L1 knockdown MDA-MB-231/LM and control (scrambled shRNA) cells, tumor growth was substantially reduced after seven days in mice xenografted with PD-L1 knockdown MDA-MB-231/LM cells (FIGS. 28C and 28D). Taken together, these results indicated that intra-tumor cell knockdown of PD-L1 functionally phenocopies important aspects of knockdown of NOTCH3, including regulation of ALDH activity and in vivo tumor growth.

TABLE 1
Oncogenic Pathways

| Description | pvalue | qvalue |
|---|---|---|
| PD-1 signaling | 2.45E−07 | 2.30E−07 |
| Translocation of ZAP-70 to Immunological | 2.45E−07 | 2.30E−07 |
| synapse | | |
| Phosphorylation of CD3 and TCR zeta chains | 1.03E−06 | 9.66E−07 |
| Generation of second messenger molecules | 1.02E−04 | 9.55E−05 |
| Interferon gamma signaling | 0.0021 | 0.0019 |
| Costimulation by the CD28 family | 0.0021 | 0.0019 |
| Adherens junctions interactions | 0.0039 | 0.0036 |
| Cell-cell junction organization | 0.0039 | 0.0036 |
| Interferon Signaling | 0.0039 | 0.0036 |
| Cell-Cell communication | 0.0042 | 0.0039 |
| Cell junction organization | 0.0044 | 0.0041 |
| Interleukin-4 and Interleukin-13 signaling | 0.0063 | 0.0059 |
| Neutrophil degranulation | 0.0114 | 0.0107 |
| MHC class II antigen presentation | 0.0215 | 0.0203 |
| Signaling by Interleukins | 0.0433 | 0.0407 |
| Extracellular matrix organization | 0.0477 | 0.0449 |
| Cell surface interactions at the vascular wall | 0.0526 | 0.0495 |

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A method for treating a mammal having a cancer identified as being resistant to a chemotherapeutic agent, wherein said method comprises:
administering a NOTCH3 inhibitor to said mammal, thereby increasing the susceptibility of said cancer to said chemotherapeutic agent; and
administering said chemotherapeutic agent to said mammal.

2. The method of claim 1, wherein said cancer is a metastatic cancer.

3. The method of claim 1, wherein said cancer comprises NOTCH3⁺ cells.

4. The method of claim 1, wherein said cancer comprises ALDH⁺ cells.

5. The method of claim 1, wherein said cancer comprises Epithelial to Mesenchymal Transition⁺ (EMT⁺) cells.

6. The method of claim 1, wherein said cancer comprises cancer stem-like cells.

7. The method of claim 1, wherein said cancer is triple negative breast cancer (TNBC), ovarian cancer, or another solid tumor with elevated NOTCH3 expression.

8. The method of claim 1, further comprising administering to said mammal a checkpoint inhibitor.

9-14. (canceled)

15. A method for monitoring treatment of a mammal having a cancer resistant to a chemotherapeutic agent, wherein said method comprises:
identifying said mammal as having an elevated level of NOTCH3 in cells from said cancer,
administering a NOTCH3 inhibitor to said mammal, and
measuring a post-treatment level of NOTCH3 in cells of said cancer after said administering, wherein a decrease in the post-treatment level of NOTCH3 as compared to said elevated level of NOTCH3 indicates successful treatment of said mammal.

16. The method of claim 15, wherein said cancer is a metastatic cancer.

17. The method of claim 15, wherein said cancer is TNBC, ovarian cancer, or another solid tumor with elevated NOTCH3 expression.

18. The method of claim 15, further comprising administering said chemotherapeutic agent to said mammal.

19. A method for treating a mammal having a cancer identified as being resistant to a chemotherapeutic agent, wherein said method comprises administering a NOTCH3 inhibitor to said mammal, thereby increasing the susceptibility of said cancer to said chemotherapeutic agent.

20. The method of claim 19, wherein said cancer is a metastatic cancer.

21. The method of claim 19, wherein said cancer comprises NOTCH3⁺ cells.

22. The method of claim 19, wherein said cancer comprises ALDH⁺ cells.

23. The method of claim 19, wherein said cancer comprises EMT⁺ cells.

24. The method of claim 19, wherein said cancer comprises cancer stem-like cells.

25. The method of claim 19, wherein said cancer is TNBC, ovarian cancer, or another solid tumor with elevated NOTCH3 expression.

26. The method of claim 19, further comprising administering to said mammal a checkpoint inhibitor.


SEQUENCE LISTING (5 sequences)

SEQ ID NO: 1 - RNA, 21 nt, synthetic construct
  source 1..21: mol_type = other RNA
atcaatgttgacttcacagtt

SEQ ID NO: 2 - RNA, 21 nt, synthetic construct
  source 1..21: mol_type = other RNA
gccagaactgtgaagtcaatt

SEQ ID NO: 3 - RNA, 24 nt, synthetic construct
  source 1..24: mol_type = other RNA
ggacatgcaggatagcaaggaggg

SEQ ID NO: 4 - RNA, 25 nt, synthetic construct
  source 1..25: mol_type = other RNA
agattaatgaggatgactgcggccc

SEQ ID NO: 5 - RNA, 25 nt, synthetic construct
  source 1..25: mol_type = other RNA
agatgggacatgttccatagccttg